(19) United States
(12) Patent Application Publication
(10) Pub. No.: US 2008/0139492 A1
(43) Pub. Date: Jun. 12, 2008

(54) Compositions and Methods That Enhance Rna Interference

(75) Inventors: Gary Ruvkun, Newton, MA (US); Scott Kennedy, Madison, WI (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 10/587,735

(22) PCT Filed: Feb. 2, 2005
(86) PCT No.: PCT/US05/02804

§ 371 Date: Aug. 6, 2007

Related U.S. Application Data
(60) Provisional application No. 60/541,223, filed on Feb. 2, 2004.

(51) Int. Cl. C12Q 1/68; G01N 33/00; C07K 14/00; C07H 21/00; C12N 15/11; A01K 67/027; A01K 67/033; A01H 5/00; C12N 5/10; C12N 15/80; C12N 15/85; C12N 15/74

(52) U.S. Cl. 514/44; 435/6; 436/501; 530/350; 536/23.1; 800/13; 800/14; 800/295; 536/24.5; 435/366; 435/455; 435/468; 435/471

(57) ABSTRACT

The invention features nucleobase oligomeric compositions useful in enhancing RNA interference in a wide variety of cell types for the treatment of virtually any disease or disorder related to the overexpression of a gene or genes, as well as methods of identifying and using such compositions.

### BACKGROUND OF THE INVENTION

In general, the invention features methods and nucleobase oligomeric compositions useful for enhancing RNA interference as well as methods for the identification of new candidate oligomeric composition for this purpose.

Exposure of many organisms to double stranded (ds) RNA causes the degradation of mRNA molecules containing sequences homologous to the trigger dsRNA. This process has been termed dsRNA-mediated interference (RNAi) in Caenorhabditis elegans, post-transcriptional gene silencing (PTGS) in plants, and quelling in fungi. RNAi is a natural defense mechanism that is thought to have evolved to protect organisms, including mammals, from viral diseases. Many viral genomes are composed of RNA. When such viruses infect a cell, they make double-stranded copies of their genetic material. Cells of many species combat such infections by targeting these dsRNAs for destruction.

dsRNAs are cleaved to small 20-25 bp interfering (si)RNAs by the RNase III enzyme dicer. These siRNAs hybridize to their cognate mRNAs, as part of a large protein complex, and induce mRNA cleavage and degradation. RNAi has been used as a tool to investigate gene function in a wide range of species. With an increasing list of genes successfully knocked-down by RNAi in mammalian cells and improvements in the delivery of siRNAs to cells, including in vivo delivery to mice, RNAi is now emerging as a therapeutic tool useful for the treatment of virtually any disease or disorder linked to the overexpression of a gene or genes. RNAi is emerging as a potent therapy for the treatment of hyperproliferative disorders (e.g., neoplasms), infectious diseases, parasites, and some dominant genetic diseases. Methods that enhance the efficiency of RNAi thus have a wide variety of clinical applications.

### SUMMARY OF THE INVENTION

As described below, the invention features nucleobase oligomeric compositions and methods useful in enhancing RNAi in a wide variety of cell types.

In one aspect, the invention generally features a method for identifying a nucleic acid molecule encoding a polypeptide that inhibits RNA interference (RNAi). The method involves providing a mutagenized nematode containing a gene that is expressed in a cell that is refractory to RNAi; contacting the nematode with an inhibitory nucleobase oligomer that targets the gene; and measuring the expression of the gene in the mutagenized nematode relative to a control nematode, where a mutation in a nucleic acid molecule encoding a polypeptide that inhibits RNAi is identified by detecting a decrease in the expression of the targeted gene. In one embodiment, the decrease is detected by monitoring the expression of a reporter gene. In another embodiment, the cell is a neuron. In yet another embodiment, the inhibitory nucleobase oligomer is a dsRNA, siRNA, or dsRNA mimetic.

In another aspect, the invention features a method for identifying a nucleic acid molecule encoding a polypeptide that inhibits RNAi. The method involves providing a mutagenized cell expressing a gene that is refractory to RNAi; contacting the cell with an inhibitory nucleobase oligomer that targets the refractory gene; and measuring the expression of the refractory gene, where a mutation in a nucleic acid molecule encoding a polypeptide that inhibits RNAi is identified by detecting the decrease. In one preferred embodiment, the cell is a nematode cell. In another preferred embodiment, the cell is a mammalian cell. In another embodiment, the decrease is detected by monitoring the expression of a reporter gene.

In another aspect, the invention features a method for identifying a candidate compound that enhances RNAi. The method involves providing a cell expressing an eri-1 nucleic acid molecule; contacting the cell with a candidate compound; and comparing the expression of the eri-1 nucleic acid molecule in the cell contacted with the candidate compound with the expression of the eri-1 nucleic acid molecule in a control cell, where a decrease in the expression identifies the candidate compound as a candidate compound that enhances RNAi. In one embodiment, the screening method identifies a compound that decreases transcription of the nucleic acid molecule. In another embodiment, the screening method identifies a compound that decreases translation of an mRNA transcribed from the nucleic acid molecule. In yet another embodiment, the compound is a member of a chemical library. In one preferred embodiment, the cell is in a nematode.

In another aspect, the invention features a method for identifying a candidate compound that enhances RNAi. The method involves providing a cell expressing an ERI-1 polypeptide; contacting the cell with a candidate compound; and comparing the biological activity of the ERI-1 polypeptide in the cell contacted with the candidate compound to a control cell, where a decrease in the biological activity of the ERI-1 polypeptide identifies the candidate compound as a candidate compound that enhances RNAi. In one embodiment, the cell is a nematode cell. In another embodiment, the cell is in a nematode. In yet another embodiment, the cell is a mammalian cell. In yet another embodiment, the ERI-1 polypeptide is an endogenous polypeptide. In one preferred embodiment, the biological activity is monitored with an enzymatic assay. In another embodiment, the biological activity is monitored with an immunological assay. In one preferred embodiment, the biological activity is monitored by detecting degradation of an ERI-1 nucleic acid substrate. In another preferred embodiment, the nucleic acid substrate is an siRNA.

In another aspect, the invention features a method for identifying a candidate compound that enhances RNAi. The method involves providing an ERI-1 polypeptide; contacting the polypeptide with a candidate compound; and detecting binding of the ERI-1 polypeptide and the candidate compound, where a compound that binds to the ERI-1 polypeptide is a candidate compound that enhances RNAi. In one preferred embodiment, the candidate compound binds to and blocks an ERI-1 active site.

In another aspect, the invention features a method for identifying a candidate compound that enhances RNAi. The method involves (a) providing an ERI-1 polypeptide and a nucleic acid substrate; (b) contacting the ERI-1 polypeptide and the nucleic acid substrate with a candidate compound under conditions suitable for substrate degradation; and (c) detecting a decrease in substrate degradation in the presence of the candidate compound relative to substrate degradation in the absence of the candidate compound, wherein a decrease in the substrate degradation identifies the candidate compound as a candidate compound that enhances RNAi. In one preferred embodiment, the nucleic acid substrate is an siRNA. In another preferred embodiment, the nucleic acid substrate is coupled to a fluorophore.

In another aspect, the invention features a method for identifying a candidate compound that enhances RNAi. The method involves (a) providing a cell expressing an ERI-1 polypeptide; (b) contacting the cell with a dsRNA in the presence of a candidate compound; and (c) monitoring a dsRNA-related phenotype in the cell in the presence of the candidate compound relative to the phenotype in the absence of the candidate compound, wherein an alteration in the phenotype identifies the candidate compound as a candidate compound that enhances RNAi.

In another aspect, the invention provides an isolated ERI-1 polypeptide containing an amino acid sequence having at least 85%, 90%, or 95% identity to the amino acid sequence of SEQ ID NO:2, where the polypeptide inhibits RNAi.

In another aspect, the invention features an isolated nucleic acid molecule containing a nucleotide sequence having at least 85%, 90%, or 95% identity to the nucleotide sequence encoding SEQ ID NO:2, where expression of the nucleic acid molecule in an organism inhibits RNAi in the organism.

In another aspect, the invention features vectors and host cells containing isolated eri-1 nucleic acid molecules and antibodies that specifically bind to ERI-1 polypeptides.

In another aspect, the invention features an organism containing a mutation in an eri-1 nucleic acid sequence, where the mutation enhances RNAi in the organism. In one embodiment, the organism is a nematode. In another embodiment, the organism is a mammal. In yet another embodiment, the organism is a plant.

In another aspect, the invention features an isolated nucleobase oligomer containing a duplex containing at least eight but no more than thirty consecutive nucleobases of an eri-1 nucleic acid, where the duplex when contacted with an eri-1 expressing cell, reduces expression of eri-1 transcription or translation. In one embodiment, the duplex contains a first domain containing between 21 and 29 nucleobases and a second domain that hybridizes to the first domain under physiological conditions, where the first and second domains are connected by a single stranded loop. In another embodiment, the loop contains between 6 and 12 nucleobases. In yet another embodiment, the loop contains 8 nucleobases. In one preferred embodiment, the oligomer reduces the level of expressed ERI-1 polypeptide.

In another aspect, the invention features a nucleobase oligomeric complex containing paired sense and antisense nucleic acid strands, where the complex contains at least eight but no more than thirty consecutive nucleobases corresponding to an eri-1 nucleic acid molecule, and the complex when contacted with an eri-1 expressing cell reduces expression of ERI-1 polypeptide. In one preferred embodiment, the nucleobase oligomeric complex is dsRNA. In one embodiment the complex contains at least one nucleic acid modification. In another embodiment, the modification is a modified sugar, nucleobase, or internucleoside linkage. In yet another embodiment, the modification is a modified internucleoside linkage selected from the group consisting of phosphorothioate, methylphosphonate, phosphotriester, phosphorodithioate, and phosphoselenate linkages. In yet another embodiment, the complex contains at least one modified sugar moiety. In a preferred embodiment, the modified nucleobase contains RNA residues. In another embodiment, the RNA residues are linked together by phosphorothioate linkages.

In another aspect, the invention features an expression vector encoding a nucleobase oligomer containing a duplex containing at least eight but no more than thirty consecutive nucleobases of an eri-1 nucleic acid, where the duplex, when contacted with an eri-1 expressing cell, reduces eri-1 transcription or translation.

In another aspect, the invention features an expression vector encoding a nucleobase oligomeric complex containing paired sense and antisense nucleic acid strands, where the complex contains at least eight but no more than thirty consecutive nucleobases corresponding to an eri-1 nucleic acid sequence, where the complex, when contacted with an eri-1 expressing cell, reduces expression of ERI-1 polypeptide. In various embodiments of the previous aspect, the nucleic acid sequence encodes a nucleobase oligomer or nucleobase oligomeric complex operably linked to a promoter. In some embodiments, the promoter is the U6 PolIII promoter, polymerase III H1 promoter. In other embodiments, a cell contains the expression vector of the previous aspects. In one preferred embodiment, the cell is a transformed human cell that stably expresses the expression vector. In other embodiments, the cell is in vivo. In another preferred embodiment, the cell is a neoplastic cell.

In another aspect, the invention features a transgenic organism expressing a nucleic acid sequence encoding an eri-1 nucleobase oligomer, where the nucleobase oligomer inhibits the expression of an endogenous eri-1 nucleic acid sequence. In one embodiment, the organism is a mammal. In another embodiment, the organism is a nematode. In yet another embodiment, the organism is a plant.

In another aspect, the invention features a method for enhancing RNAi in an organism, the method involves contacting the organism with a nucleobase oligomer of any previous aspect in an amount sufficient to enhance RNAi. In various embodiments, the organism is a plant, a mammal, or a pathogen (e.g., a bacteria, a virus, a fungus, an insect, or a nematode). In preferred embodiments, the nucleobase oligomer is an siRNA or an shRNA.

In another aspect, the invention features a pharmaceutical composition containing an eri-1 nucleobase oligomer and an excipient.

In another aspect, the invention features a double-stranded RNA corresponding to at least a portion of an eri-1 nucleic acid molecule of an organism, where the double-stranded RNA is capable of decreasing the level of ERI-1 polypeptide encoded by an eri-1 nucleic acid molecule.

In another aspect, the invention features an antisense nucleic acid molecule, where the antisense nucleic acid molecule is complementary to at least twelve nucleotides of an eri-1 nucleic acid molecule, and where the antisense nucleic acid molecule is capable of decreasing expression of an ERI-1 polypeptide from an eri-1 nucleic acid molecule.

In another aspect, the invention features a method for identifying an siRNA having enhanced RNAi activity, the method involving contacting a test siRNA with an ERI-1 polypeptide under conditions suitable for RNA degradation; and detecting an increased amount of undegraded test siRNA relative to a control siRNA known to be degraded under similar conditions, where increased resistance to degradation indicates that the test siRNA has enhanced RNAi activity.

In another aspect, the invention features an siRNA capable of inducing enhanced RNAi, the siRNA containing a 3′ terminus having 2, 3, 4, or 5 cytosine bases or guanine bases, such that the siRNA resists degradation by ERI-1.

In another aspect, the invention features a method for preventing or ameliorating a disease in an organism, the method involving contacting the organism with an eri-1 inhibitory nucleobase oligomer and with a nucleobase oligomer that interferes with the expression of a target gene expressed in the disease. In one embodiment, the eri-1 inhibitory nucleobase oligomer enhances RNAi of the target gene. In another embodiment, the target gene is an endogenous gene of the organism. In another embodiment, the target gene is expressed in a pathogen. In yet another embodiment, the disease is a neoplasm. In other embodiments, the disease is a bacterial, viral, or parasitic infection.

In another aspect, the invention features a method for preventing or ameliorating a disease in an organism. The method involves contacting the organism with an eri-1 inhibitory nucleobase oligomer and with a nucleobase oligomer that interferes with the expression of a target gene expressed in the disease. In one embodiment, the eri-1 inhibitory nucleobase oligomer enhances RNAi of the target gene. In another embodiment, the target gene is an endogenous gene of the organism. In yet another embodiment, the target gene is expressed in a pathogen. In another embodiment, the disease is a neoplasm. In still other embodiments, the disease is a bacterial, viral, or parasitic infection.

In various embodiments of any of the above aspects, an inhibitory nucleobase oligomer (e.g., antisense nucleobase oligomer, dsRNA, siRNA, or shRNA) comprises at least 12, 15, 16, 17, 18, 19, 20, 21, 22, 23, 24, or 25 nucleobases complementary to an eri-1 nucleic acid molecule.

In other preferred embodiments of any of the previous aspects, the naturally occurring eri-1 nucleic acid molecule is T07A9.5, BC035279, T04799, AP000815.1, AP003103.2, AK120298.1, NM_{—}191971.1, or AY112398.1. In other preferred embodiments of any of the previous aspects, the naturally occurring eri-1 nucleic acid molecule is an eri-1 ortholog that encodes a polypeptide selected from the group consisting of any one or all of the following BC035279, BAB02568.1, NP_{—}566502.1, T04799, NP_{—}921413.1, NP_{—}179108.1, AAL31944.1, AAL84996.1, CAB36522.1, CAB79531.1, AAK98687.1, AAP53700.1, NP_{—}499887.1, NP_{—}500418.1, NP_{—}741292.1, NP_{—}741293.1, T28707, NP_{—}508415.1, NP_{—}497750.1, NP_{—}507742.1, T15066, AAB94148.1, T29900, AAB09126.1, AAK39277.2, NP_{—}741293.1, T32575, AAK39278.1, T28707, NP_{—}508415.1, Q10905, YWO2_CAEEL, T30086, AAA82440.1, AAP57300.1, NP_{—}741293.1, NP_{—}507945.1, T19258, NP_{—}505050.1, T32575, AAK39278.1, T26693, CAA20983.1, T33294, AAC17749.1, AK064632.1, AP002897.2, AK103348.1, AK062026.1, AY105868.1, NM_{—}112377.1, AF419612.1, AF419612, AY079112.1, AP002862.2, AP000815.1, AP003103.2, AK120298.1, NM_{—}191971.1, AY112398.1, AC146855.5, AY105981.1, NM_{—}117213.2, AF291711.1, AF291711, AK120333.1, AK106560.1, AB019236.1, AK122166.1, NM_{—}184142.1, NM_{—}196431.1, and AC093544.8.

In other aspects, the invention generally features an isolated eri-1 inhibitory nucleic acid comprising at least a portion of a naturally occurring eri-1 nucleic acid molecule of an organism, or its complement, where the eri-1 nucleic acid encodes a polypeptide selected from the group consisting of any or all of the following T07A9.5, BC035279, T04799, BC035279, BAB02568.1, NP_{—}566502.1, T04799, NP_{—}921413.1, NP_{—}179108.1, AAL31944.1, AAL84996.1, CAB36522.1, CAB79531.1, AAK98687.1, AAP53700.1, NP_{—}499887.1, NP_{—}500418.1, NP_{—}741292.1, NP_{—}741293.1, T28707, NP_{—}508415.1, NP_{—}497750.1, NP_{—}507742.1, T15066, AAB94148.1, T29900, AAB09126.1, AAK39277.2, NP_{—}741293.1, T32575, AAK39278.1, T28707, NP_{—}508415.1, Q10905, YWO2_CAEEL, T30086, AAA82440.1, AAP57300.1, NP_{—}741293.1, NP_{—}507945.1, T19258, NP_{—}505050.1, T32575, AAK39278.1, T26693, CAA20983.1, T33294, AAC17749.1, AK064632.1, AP002897.2, AK103348.1, AK062026.1, AY105868.1, NM_{—}112377.1, AF419612.1, AF419612, AY079112.1, AP002862.2, AP000815.1, AP003103.2, AK120298.1, NM_{—}191971, AY112398.1, AC146855.5, AY105981.1, NM_{—}117213.2, AF291711.1, AF291711, AK120333.1, AK106560.1, AB019236.1, AK122166.1, NM_{—}184142.1, NM_{—}196431.1, and AC093544.8, or an ortholog of any or all of these eri-1 nucleic acid molecules, where the eri-1 inhibitory nucleic acid contains at least a portion of a naturally occurring eri-1 nucleic acid molecule, or is capable of hybridizing to a naturally occurring eri-1 nucleic acid molecule, and decreases expression from a naturally occurring eri-1 nucleic acid molecule in the organism.

In preferred embodiments of the above aspects, an eri-1 nucleic acid is any one or all of the following or a portion thereof, or an ortholog of any or all of these nucleic acids: T07A9.5, BC035279, T04799, BC035279, BAB02568.1, NP_{—}566502.1, T04799, NP_{—}921413.1, NP_{—}179108.1, AAL31944.1, AAL84996.1, CAB36522.1, CAB79531.1, AAK98687.1, AAP53700.1, NP_{—}499887.1, NP_{—}500418.1 NP_{—}741292.1, NP_{—}741293.1, T28707, NP_{—}508415.1, NP_{—}497750.1, NP_{—}507742.1, T15066, AAB94148.1, T29900, AAB09126.1, AAK39277.2, NP_{—}741293.1, T32575, AAK39278.1, T28707, NP_{—}508415.1, Q10905, YWO2_CAEEL, T30086, AAA82440.1, AAP57300.1, NP_{—}741293.1, NP_{—}507945.1, T19258, NP_{—}505050.1, T32575, AAK39278.1, T26693, CAA20983.1, T33294, AAC17749.1, AK064632.1, AP002897.2, AK103348.1, AK062026.1, AY105868.1, NM_{—}112377.1, AF419612.1, AF419612, AY079112.1, AP002862.2, AP000815.1, AP003103.2, AK120298.1, NM_{—}191971, AY112398.1, AC146855.5, AY105981.1, NM_{—}117213.2, AF291711.1, AF291711, AK120333.1, AK106560.1, AB019236.1, AK122166.1, NM_{—}184142.1, NM_{—}196431.1, and AC093544.8.

By “eri-1 nucleic acid molecule” is meant a polynucleotide sequence having at least 85% amino acid identity to C. elegans eri-1 (T07A9.5 (present in GenBank Accession No. AF036706)), human eri-1 (GenBank Accession No. BC035279), or Arabidopsis eri-1 (T04799), or hybridizing under stringent conditions to T07A9.5, or GenBank Accession Nos. BC035279 or T04799, and encoding a gene product having nuclease activity. Preferably, an eri-1 nucleic acid encodes a polypeptide having at least 85%, more preferably at least 90%, and most preferably at least 95% identity to a T07A9.5, BC035279, or T04799 exonuclease domain. Optionally, the encoded polypeptide further contains a SAP domain N-terminal to a DEDDh nuclease domain.

By “ERI-1 polypeptide” is meant a protein, or fragment thereof, having at least 85% amino acid identity to a protein encoded by T07A9.5 (e.g., GenBank Accession Nos. AAK39277 and AAK39278), GenBank Accession No. BC035279, or T04799 and having nuclease activity. Optionally, an ERI-1 polypeptide further contains a SAP domain N-terminal to a DEDDh nuclease domain. Examples of ERI-1 polypeptides include Caenorhabditis briggsae (Cb) (AC084443), Homo sapiens (Hs) GenBank Accession No: AAH35279) (also termed 3′ hExo), Mus musculus (Mm) GenBank Accession No: NM_{—}026067, the Danio rerio (Dr) polypeptide encoded by a nucleic acid molecule constructed by fusion of GenBank Accession Nos: BQ285328 and BI888174, and Schizosaccharomyces pombe (Sp) GenBank Accession No: NP_{—}595533.

By “ERI-1 ortholog” is meant a protein, or fragment thereof, that is highly related to an ERI-1 polypeptide and that has nuclease activity. A “highly related sequence” corresponds to a candidate ERI-1 ortholog identified using a tblastn search executed with an ERI-1 polypeptide as the reference sequence, where the probability that the candidate would be randomly identified is less than e⁻³, e⁻⁵, e⁻¹⁰, or e⁻²⁰. Such candidates are retrieved from Genbank (http://www.ncbi.nlm.nih.gov/) and verified by using the candidate sequence as a reference sequence in a BLASTp search of C. elegans proteins (e.g., wormbase site (http://www.wormbase.org/db/searches/blast)), where the search identifies the original C. elegans sequence as a highly related sequence.

Candidate ERI-1 orthologs identified using such methods include, but are not limited to, BC035279, BAB02568.1, NP_{—}566502.1, T04799, NP_{—}921413.1, NP_{—}179108.1, AAL31944.1, AAL84996.1, CAB36522.1, CAB79531.1, AAK98687.1, AAP53700.1, NP_{—}499887.1, NP_{—}500418.1, NP_{—}741292.1, NP_{—}741293.1, T28707, NP_{—}508415.1 NP_{—}497750.1, NP_{—}507742.1, T15066, AAB94148.1, T29900, AAB09126.1, AAK39277.2, NP_{—}741293.1, T32575, AAK39278.1, T28707, NP_{—}508415.1, Q10905, YWO2_CAEEL, T30086, AAA82440.1, AAP57300.1, NP_{—}741293.1, NP_{—}507945.1, T19258, NP_{—}505050.1, T32575, AAK39278.1, T26693, CAA20983.1, T33294, AAC17749.1, AK064632.1, AP002897.2, AK103348.1, AK062026.1, AY105868.1, NM_{—}112377.1, AF419612.1, AF419612, AY079112.1, AP002862.2, AP000815.1, AP003103.2, AK120298.1, NM_{—}191971.1, AY112398.1, AC146855.5, AY105981.1, NM_{—}117213.2, AF291711.1, AF291711, AK120333.1, AK106560.1, AB019236.1, AK122166.1, NM_{—}184142.1, NM_{—}196431.1, and AC093544.8.

Such candidate ERI-1 orthologs are assayed for nuclease activity using methods described, for example, by Dominski et al., (Mol Cell 12:295-305, 2003).

By “the biological activity of an ERI-1 polypeptide” is meant nuclease activity. One example of nuclease activity is RNAse activity. A compound that enhances RNAi would be expected to decrease ERI-1 biological activity by at least 10%, 25%, 50%, 75%, or even by at least 80% or 90%.

By “anti-sense” is meant a nucleic acid sequence, regardless of length, that is complementary to the coding strand or mRNA of a nucleic acid sequence. Desirably the anti-sense nucleic acid is capable of decreasing the expression or biological activity of a nucleic acid or amino acid sequence. In a desirable embodiment, the decrease in expression or biological activity is at least 10%, relative to a control, more desirably 25%, and most desirably 50% or more. The anti-sense nucleic acid may contain a modified backbone, for example, phosphorothioate, phosphorodithioate, or other modified backbones known in the art, or may contain non-natural internucleoside linkages.

“Cell” as used herein may be a single-cellular organism, cell from a multi-cellular organism, or it may be a cell contained in a multi-cellular organism.

By “derived from” is meant isolated from or having the sequence of a naturally occurring sequence (e.g., a cDNA, genomic DNA, synthetic, or combination thereof).

By “differentially expressed” is meant having a difference in the expression level of a nucleic acid or polypeptide. This difference may be either an increase or a decrease in expression, when compared to control conditions.

By “double stranded RNA” is meant a complementary pair of sense and antisense RNAs regardless of length. In one embodiment, these dsRNAs are introduced to an individual cell, tissue, organ, or to a whole animals. For example, they may be introduced systemically via the bloodstream. Desirably, the double stranded RNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more.

By “duplex” is meant a domain containing paired sense and antisense nucleobase oligomeric strands. For example, a duplex comprising 29 nucleobases contains 29 nucleobases on each of the paired sense and antisense strands.

By “hybridize” is meant pair to form a double-stranded complex containing complementary paired nucleobase sequences, or portions thereof, under various conditions of stringency. (See, e.g., Wahl, G. M. and S. L. Berger (1987) Methods Enzymol. 152:399; Kimmel, A. R. (1987) Methods Enzymol. 152:507) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York. Preferably, hybridization occurs under physiological conditions. Typically, complementary nucleobases hybridize via hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immunological assay” is meant an assay that relies on an immunological reaction, for example, antibody binding to an antigen. Examples of immunological assays include ELISAs, Western blots, immunoprecipitations, and other assays known to the skilled artisan.

By an “inhibitory nucleobase oligomer” is meant a dsRNA, siRNA, shRNA, or mimetic thereof that inhibits the expression of a target gene (e.g., an eri-1 gene or a gene of interest). An inhibitory nucleobase oligomer typically reduces the amount of a target mRNA, or protein encoded by such mRNA, by at least 5%, more desirable by at least 10%, 25%, 50%, or even by 75%, 85%, or 90% relative to an untreated control. Methods for measuring both mRNA and protein levels are well-known in the art; exemplary methods are described herein.

Preferably, an inhibitory nucleobase oligomer of the invention is capable of enhancing RNAi by decreasing eri-1 mRNA or protein levels. Preferably a nucleobase oligomer of the invention includes from about 8 to 30 nucleobases. A nucleobase oligomer of the invention may also contain, for example, an additional 20, 40, 60, 85, 120, or more consecutive nucleobases that are complementary to an eri-1 polynucleotide. The nucleobase oligomer (or a portion thereof) may contain a modified backbone. Phosphorothioate, phosphorodithioate, and other modified backbones are known in the art. The nucleobase oligomer may also contain one or more non-natural linkages.

By “isolated polynucleotide” is meant a nucleic acid (e.g., a DNA) that is free of the genes that, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “mutagenized” is meant comprising a mutation. Mutations may be naturally occurring or induced by contacting a cell or organism with any agent that induces a break or alteration in a nucleic acid, preferably a genomic nucleic acid. Such agents are known to the skilled artisan and include radiation (e.g., U.V., gamma, and X-rays) and chemical agents (e.g., ethylmethanesulfonate (EMS), aflatoxin B₁, nitrosoguanidine).

“Microarray” means a collection of nucleic acid molecules or polypeptides from one or more organisms arranged on a solid support (for example, a chip, plate, or bead). These nucleic acid molecules or polypeptides may be arranged in a grid where the location of each nucleic acid molecule or polypeptide remains fixed to aid in identification of the individual nucleic acid molecules or polypeptides. A microarray may include, for example, nucleic acid molecules representing all, or a subset, of the open reading frames of an organism, or of the polypeptides that those open reading frames encode. A microarray may also be enriched for a particular type of gene.

By “nucleic acid” is meant an oligomer or polymer of ribonucleic acid or deoxyribonucleic acid, for example, a dsRNA, siRNA, shRNA, or mimetic thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

Specific examples of some preferred modified nucleic acids or nucleobases envisioned for this invention may contain phosphorothioates, phosphotriesters, methyl phosphonates, short chain alkyl or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Also preferred are oligonucleotides having morpholino backbone structures (Summerton, J. E. and Weller, D. D., U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen, M. Egholm, R. H. Berg, O Buchardt, Science 199, 254, 1497). Other preferred oligonucleotides may contain alkyl and halogen-substituted sugar moieties comprising one of the following at the 2′ position: OH, SH, SCH₃, F, OCN, O(CH₂)ₙNH₂ or O(CH₂)ₙ CH₃, where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group.

Other preferred embodiments may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines. Each of the above is referred to as a “modification” herein.

By a nucleobase oligomer that “reduces the expression” of a target gene is meant one that decreases the amount of a target mRNA, or protein encoded by such mRNA, by at least about 5%, more desirable by at least about 10%, 25%, or even 50%, relative to an untreated control. Methods for measuring both mRNA and protein levels are well-known in the art; exemplary methods are described herein. Preferably, a nucleobase oligomer of the invention is capable of enhancing RNA interference.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “ortholog” is meant a polypeptide or nucleic acid molecule of an organism that is highly related to a reference protein, or nucleic acid sequence, from another organism. An ortholog is functionally related to the reference protein or nucleic acid sequence. In other words, the ortholog and its reference molecule would be expected to fulfill similar, if not equivalent, functional roles in their respective organisms. It is not required that an ortholog, when aligned with a reference sequence, have a particular degree of amino acid sequence identity to the reference sequence. A protein ortholog might share significant amino acid sequence identity over the entire length of the protein, for example, or, alternatively, might share significant amino acid sequence identity (e.g., at least 20%, 25%, 30%, 40%, more preferably, at least 50%, 60%, 75%, or most preferably, at least 85%, 90%, or 95%) over only a single functionally important domain of the protein. Such functionally important domains may be defined by genetic mutations or by structure function assays. Orthologs may be identified using methods provided herein. The functional role of an ortholog may be assayed using methods well known to the skilled artisan, and described herein. For example, function might be assayed in vivo or in vitro using a biochemical, immunological, or enzymatic assays; transformation rescue, or in a bioassay for the effect of gene inactivation on nematode phenotype as described herein. Alternatively, bioassays may be carried out in tissue culture; function may also be assayed by gene inactivation (e.g., by RNAi, siRNA, or gene knockout), or gene over-expression, as well as by other methods.

By “pathogen” is meant a bacteria, virus, fungus, nematode, insect, tick, arachnid or other creature which is capable of infecting or infesting host, and in particular, a plant or vertebrate animal.

By “polypeptide” is meant any chain of amino acids, or analogs thereof, regardless of length or post-translational modification (for example, glycosylation or phosphorylation).

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “promoter” is meant a polynucleotide sufficient to direct transcription.

By “purified antibody” is meant an antibody that is at least 60%, by weight, free from proteins and naturally occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinant polypeptide of the invention and standard techniques.

By “refractory to RNAi” is meant a cell or gene that is resistant to the gene silencing effects of an inhibitory nucleic acid. Cells and genes that are refractory to RNAi fail to exhibit at least a 10%, 25%, 50%, or 75% decrease in the level of expression of a gene targeted for RNAi relative to the level of the target gene's expression present in an untreated control cell.

By “reporter gene” is meant a gene encoding a polypeptide whose expression may be assayed; such polypeptides include, without limitation, -glucuronidase (GUS), luciferase, chloramphenicol transacetylase (CAT), and beta-galactosidase.

By “specifically binds” is meant a compound or antibody which recognizes and binds a polypeptide of the invention but which does not substantially recognize and bind other molecules in a sample, for example, a biological sample, which naturally includes a polypeptide of the invention.

By “shRNA” is meant an RNA comprising a duplex region complementary to an mRNA. For example, a short hairpin RNA (shRNA) may comprise a duplex region containing nucleoside bases, where the duplex is between 17 and 29 bases in length, and the strands are separated by a single-stranded 4, 5, 6, 7, 8, 9, or 10 base linker region. Optimally, the linker region is 6 bases in length.

By “siRNA” is meant a double stranded RNA comprising a region of an mRNA. Optimally, an siRNA is 17, 18, 19, 20, 21, 22, 23, or 24 nucleotides in length and has a 2 base overhang at its 3′ end. siRNAs can be introduced to an individual cell, tissue, organ, or to a whole animals. For example, they may be introduced systemically via the bloodstream. Such siRNAs are used to downregulate mRNA levels or promoter activity. Desirably, the siRNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The siRNA may contain a modified backbone, for example, phosphorothioate, phosphorodithioate, or other modified backbones known in the art, or may contain non-natural internucleoside linkages. Such siRNAs are used to downregulate mRNA levels or promoter activity.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90% or even 95% identical at the amino acid level or nucleic acid level to the sequence used for comparison. The comparison is over at least 25-50 nucleotides, more preferably 50-100 or 100-200 nucleotides, and most preferably 200-400, 400-600, 600-800, or even 800-1000 nucleotides.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “targets a gene” means specifically binds to and decreases the expression of the gene. For example, an inhibitory nucleic acid binds to and decreases the expression of a complementary target gene. Such a decrease is by at least 10%, 25%, 50%, 75%, or 100% relative to the expression of a corresponding control gene.

By “transformed cell” is meant a cell into which (or into an ancestor of which) has been introduced, by means of recombinant DNA techniques, a polynucleotide molecule encoding (as used herein) a polypeptide of the invention.

By “transgene” is meant any piece of DNA that is inserted by artifice into a cell and becomes part of the genome of the organism that develops from that cell or, in the case of a nematode transgene, becomes part of a heritable extrachromosomal array. Such a transgene may include a gene which is partly or entirely heterologous (i.e., foreign) to the transgenic organism, or may represent a gene homologous to an endogenous gene of the organism.

By “transgenic” is meant any cell which includes a DNA sequence which is inserted by artifice into a cell and becomes part of the genome of the organism which develops from that cell or part of a heritable extrachromasomal array. As used herein, the transgenic organisms are generally transgenic invertebrates, such as C. elegans, or vertebrates, such as, zebrafish, mice, and rats, and the DNA (transgene) is inserted by artifice into the nuclear genome or into a heritable extrachromasomal array.

The invention provides methods and compositions that are useful for enhancing RNAi. In addition, the methods of the invention provide a facile means to identify therapies that are safe for use in eukaryotic host organisms (i.e., compounds that do not adversely affect the normal development, physiology, or fertility of the organism). Other features and advantages of the invention will be apparent from the detailed description, and from the claims.

### BRIEF DESCRIPTION OF THE DRAWINGS

FIGS. 1A-1D are photomicrographs showing the effects of GFP dsRNA on wild-type (FIGS. 1A and 1B) and eri-1(mg366) (FIGS. 1C and 1D) L2 worms carrying an integrated unc-47::GFP transgene. These worms were grown on bacteria expressing either vector control or dsRNA derived from GFP. Fluorescent microscopy was performed on representative L4 progeny.

FIGS. 2A-2D are photomicrographs showing that eri-1 animals show enhanced sensitivity to GFP dsRNA. Wild-type (FIGS. 2A and 2B) and eri-1(mg366) (FIGS. 2C and 2D) L2 animals carrying an integrated tub-1::GFP transgene were grown on bacteria expressing either vector control or dsRNA derived from GFP. Fluorescent microscopy was performed on representative L4 progeny.

FIG. 3A is a schematic illustration of eri-1. SAP/SAF-BOX and DEDDh 3′-5′ exonuclease domains are shown. The locations of mg366 and mg388 lesions are indicated. Due to a direct repeat the exact sequence of the 23 base pair insertion in mg366 is unknown; however, it includes 23 of these 32 nucleotides ttcgataaagtgcctgtttttttttgataaa (SEQ ID NO:6). mg388 is a G to A transition at nucleotide position 35375 of cosmid T07A9. TblastN analysis identified one gene in Caenorhabditis briggsae (Cb) (AC08443), Homo sapiens (Hs) (AAH35279) (also termed 3′ hExo), Mus musculus (Mm)(NM_{—}026067), Danio rerio (Dr)(constructed by fusion of BQ285328 and BI888174), and Schizosaccharomyces pombe (Sp) (NP_{—}595533), containing a predicted SAP domain N-terminal to a predicted DEDDh nuclease domain.

FIG. 3B is a phylogenetic analysis of the eri-1 family of nucleases. Nuclease domains of eri-1 family and 12 additional randomly chosen DEDDh nucleases were aligned with the Clustal program. Members of the eri-1 family of nucleases are indicated with underlining. The nuclease domain of a Xenopus laevis (Xl) 5′ truncated EST (AW199662) containing a DEDDh domain and a 5′ truncated sap domain was included in this analysis. Shown in black are the 12 randomly chosen DEDDh nucleases; accession numbers are indicated, Arabidopsis thaliana (At) and Drosophila melanogaster (Dm). An alignment generated using all Hs, Dm, Ce and Sp Deddh nuclease domains gave similar results.

FIG. 3C is an alignment of eri-1 and other members of the DEDDh family of nucleases (SEQ ID NOS:12, 13, 14, 15, 16, 17, 18, and 19). This alignment was generated by the Clustal program. Shown are three conserved motifs (I, II, and III) found within the approximately 200 amino acid nuclease domain of the DEDDh superfamily of nucleases. Asterisks indicate highly conserved active site residues. Dark shading within motifs I and III indicates highly conserved residues within the DEDDh superfamily of nucleases. Lighter shading within motif II indicates substituted residues within the eri-1 DEMDh subfamily of nucleases. A randomly chosen DEDDh nuclease domain (AAH051864) was included for reference purposes.

FIG. 4 is a Northern blot analysis of RNA from wild-type and eri-1(mg366) eggs showing that eri-1 is differentially spliced. The nuclease domain of eri-1 was used as a probe for detecting eri-1 message. The two splice variants of eri-1, eri-1a, and eri-1b, are indicated. Molecular weight markers are indicated. eri-1 message is absent in RNA prepared from eri-1(mg366) animals likely due to nonsense mediated decay. Background fluorescence in the region between eri-1a and eri-1b coincides with ribosomal RNA. Ethidium bromide stained ribosomal RNA is shown as a loading control.

FIG. 5A is an autoradiogram showing that siRNAs are more abundant and stable in eri-1 mutant animals than in wild-type animals. Twenty wild-type and twenty eri-1(mg366) animals were injected with 5 mg/ml 5′ end labelled unc-22 ds siRNAs. Preinjected siRNAs (lane 1), RNA prepared from injected P0s (lanes 2 and 3), and RNA prepared from progeny (lanes 4 and 5) were run on an SDS PAGE gel. The blot was then probed for U6 RNA as a loading control. These results are representative of four independent experiments. We also observed slowly migrating radioactivity (presumably reincorporated phosphate) that in three of four experiments was more pronounced in eri-1 progeny than in wild-type progeny.

FIG. 5B shows an RNase protection assay performed on wild-type, eri-1(mg366), rrf-3(pk1426), and rde-1(ne300) animals grown on E. coli (HT115) bacteria containing a control vector or HT115 bacteria expressing pos-1 dsRNA diluted 10× (to maximize the difference in siRNA abundance between wild-type and eri-1 animals) with HT115. After 14 hours animals were harvested and RNA was isolated. RNA was incubated with apos-1 coding strand RNA probe. Protected RNAs were run on a 15% PAGE gel. An 18 nucleotide RNA was run as a molecular weight marker. One picomole of a synthetic antisense pos-1 siRNA was included as a control. Also shown are rRNA band from an agarose gel run in parallel. Results are representative of two independent experiments. It is likely that the amount of siRNA generated by wild-type animals feeding on pos-1 dsRNAs induces sufficient degradation of the pos-1 mRNA to cause an embryonic lethal phenotype because eri-1(mg366) does not exhibit an enhanced lethal phenotype nor enhanced kinetics of pos-1 mRNA degradation following exposure to pos-1 dsRNA.

FIG. 6A is an autoradiogram showing that ERI-1 is an siRNase. Twenty wild-type and twenty eri-1(mg366) animals were injected with 5 mg/ml of 5′ gamma P³² end labelled unc-22 ds siRNAs. Preinjected siRNAs (lane 1), RNA prepared from injected P0s (lanes 2 and 3), and RNA prepared from the progeny of these P0s (lanes 4 and 5) were run on a PAGE gel. The blot was probed for U6 RNA as a loading control. Results are representative of four independent experiments. We also observed slowly migrating bands (presumably reincorporated P³²) that in three of four experiments were more pronounced in eri-1 progeny than in wild-type progeny (data not shown).

FIG. 6B shows an RNase protection assay performed on wild-type, eri-1(mg366), rrf-3(pk1426), and rde-1(ne300) animals grown on HT115 bacteria containing a control vector or HT115 bacteria expressing pos-1 dsRNA diluted ten times with HT115 for 14 hours. Purified RNA was incubated with apos-1 coding strand RNA probe. An 18 nucleotide RNA was run as a molecular weight marker. One picomole of a synthetic antisense pos-1 siRNA was included as an RNAse protection control. Also shown is an rRNA band from an agarose gel run in parallel. Results are representative of two independent experiments. It is likely that the amount of siRNA generated by wild-type animals feeding on pos-1 dsRNAs induces sufficient degradation of the pos-1 mRNA to cause an embryonic lethal phenotype because eri-1(mg366) does not exhibit an enhanced lethal phenotype nor enhanced kinetics of pos-1 mRNA degradation following exposure to pos-1 dsRNA (data not shown).

FIG. 6C is an autoradiogram showing that Ce ERI-1 and Hs ERI1 are sufficient to degrade 3′ overhangs of ds siRNAs. eri-1A and full length human eri-1 cDNAs were appended with a C-terminal T7 promoter and an N-terminal FLAG epitope tag was transcribed in vitro. Ce ERI, hs ERI-1 mRNAs, or water (control) was added to reticulocyte lysates (AMBION #1200) and the mixture was immunoprecipitated in 50 mM Tris [pH 8.0], 200 mM NaCl, 1 mM DTT, and 0.5% NP-40 with α-FLAG conjugated agarose beads (SIGMA) and eluted with a FLAG peptide. S³⁵ methionine-labelled reticulolysates with Ce ERI and hs ERI-1 were run on a protein gel and gave a single predominant band of the expected molecular weights (data not shown). Eluates were incubated as described by Dominski et al., (Mol Cell 12:295-305, 2003) with single-strand unc-22 siRNA (lanes 1-4), double-strand unc-22 siRNA containing 2 nucleotide 3′ overhangs (lanes 5-8 and 13-15), and unc-22 siRNA hybridized to a 220 nucleotide unc-22 RNA (lanes 9-12) for 30 minutes at 37° (lanes 1-12) or 1 hour at 37° (lane 13 and 15) or 1 hour at 22° (lane 14). 5′ end labelled (*) siRNA are shown.

FIG. 7 is a series of photomicrographs showing DAPI fluorescence in wild-type and eri-1 mutant animals that exhibit a morphological defect in sperm formation. eri-1(mg366) and wild-type animals were grown at the non-permissive temperature (25° C.) and young adult animals were fixed and stained with DAPI. Arrows indicate a normal sperm nuclei in panel labelled wild-type and abnormal sperm nuclei in panel labelled eri-1(mg366). Not shown, some eri-1 sperm are abnormally small while others appear to contain two fused nuclei. The overall penetrance of gross morphological sperm defects is 25%.

FIGS. 8A-8C are photomicrographs showing that ERI-1 is expressed in the cytoplasm of a subset of head and tail neurons and also in the spermatheca. In all panels dorsal is up and anterior is to the left. FIG. 8A shows expression of the full length eri-1b::GFP in a subset of head and tail neurons (including axonal projections) and in the developing gonad in a late L2 larva. The inset panel is a magnification of GFP expressing head neurons. FIG. 8B shows that in adult animals eri-1b::GFP gonadal expression is restricted to the spermatheca. GFP fluorescence in the anterior aspect of the posterior spermatheca is shown. FIG. 8C shows that the transcriptional fusion eri-1p::GFP is most prominently expressed in a subset of head and tail neurons and is also expressed at a low level ubiquitously.

FIGS. 9A and 9B show the nucleic acid (SEQ ID NO:1) and amino acid (SEQ ID NOS:2 and 3) sequences of C. elegans eri-1.

FIGS. 10A and 10B show the nucleic acid (SEQ ID NO:4) and amino acid (SEQ ID NO:5) sequences of human eri-1.

FIG. 10C shows the amino acid sequences of human, C. elegans, rice, maize, and Arabidopsis ERI-1 nuclease domains (SEQ ID NOS:7, 8, 9, 10, and 11).

FIG. 11 is an autoradiogram showing that siRNAs containing five cyosine or guanosine bases were resistant to ERI-1 nuclease activity.

FIGS. 12A-12D are a series of photomicrographs showing the effect of dpy-13 RNAi on wild-type (FIG. 12A), eri-1(mg366) (FIG. 12B), dpy-13(e458) (FIG. 12C), and eri-1 (mg366); dpy-13(e458) (FIG. 12D) nematodes. The left panel in each of FIGS. 12A-12D shows an untreated control nematode of the indicated genotype.

### DESCRIPTION OF THE INVENTION

The present invention features methods and compositions useful for enhancing RNAi in a wide variety of cell types.

This invention derives, at least in part, from Applicants' discovery of a siRNAse, C. elegans eri-1, which was isolated in a genetic screen for mutants that show enhanced RNAi. Inhibition of human or plant eri-1 transcription or translation or inhibition of ERI-1 biological activity facilitates the more effective use of RNAi-based therapies. Accordingly, the invention features in vitro and in vivo screening methods for the identification of compounds that inhibit the nuclease activity of ERI-1 polypeptides. In addition, the invention provides for eri-1 inhibitory nucleic acids and methods of using such compounds.

eri-1 mutant animals were more sensitive than wild-type animals to RNAi induced by feeding on E coli expressing dsRNA or injection of siRNAs derived from a broad set of genes. Genetic analysis placed eri-1 either upstream or in parallel to the RNAi defective mutants rde-1, rde-4, sid-1, and mut-16 and in the same genetic pathway with the RNA dependent RNA polymerase gene rrf-3. eri-1 encodes a conserved protein with DEDDh 3′-5′ exonuclease and SAP/SAF-BOX domains. eri-1 mutant animals accumulated more siRNAs than wild-type following exposure to dsRNA. In addition, siRNAs exhibited an extended half-life in eri-1 animals consistent with ERI-1 functioning to degrade siRNAs. ERI-1 was expressed predominantly within a subset of head and tail neurons and localized to the cytoplasm.

eri-1 enhanced neuronal susceptibility to RNAi

In C. elegans dsRNAs vary in their ability to trigger efficient RNAi. RNAi in C. elegans can be induced by feeding on bacteria expressing dsRNA, but dsRNAs vary in their ability to trigger efficient RNAi (Timmons et al., Gene 263:103-12, 2001). Feeding-induced RNAi of about 65% of the genes defined by classical genetic analysis causes a phenotype similar to that predicted from the loss of function (loƒ) mutant phenotype (Fraser et al., Nature 408:325-30, 2000; Kamath et al., Nature 421:231-7, 2003). Interestingly, mRNAs expressed within the nervous system are refractory to RNAi. For example, nearly all neuronally expressed genes that are known to be mutable towards an uncoordinated (Unc) phenotype are resistant to RNAi (Timmons et al., Gene 263:103-12, 2001; Tavernarakis et al., Nat Genet. 24:180-3 2000; Fraser et al., Nature 408:325-30, 2000). While genetic screens have identified components required for RNAi (Hannon, Nature 418:244-51, 2002), little is known about negative regulators of RNAi that may explain why a subset of mRNAs and cell types are refractory to RNAi, or how episodes of RNAi are resolved.

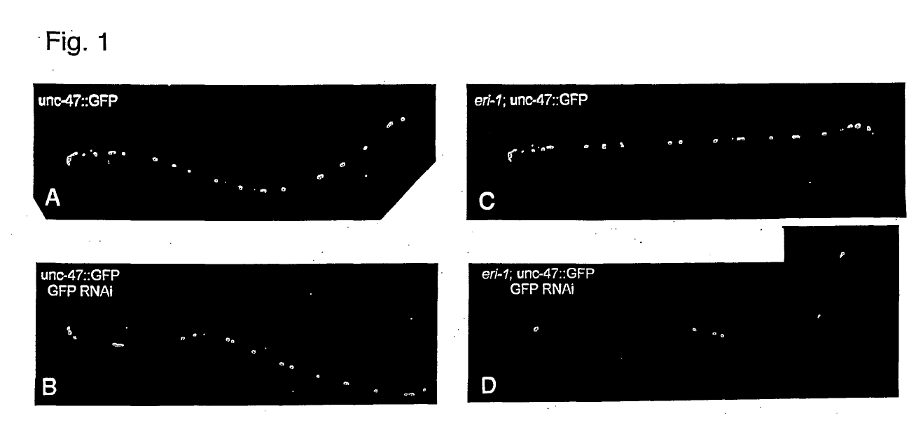

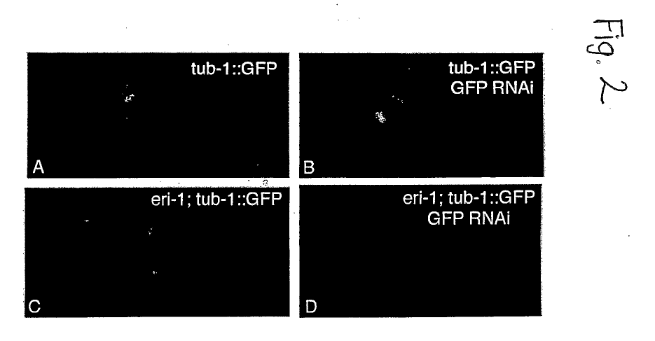

We took advantage of the relative inefficiency of neuronal RNAi in C. elegans and performed a genetic screen for mutants with enhanced sensitivity to dsRNAs. Such genes are expected to normally inhibit the uptake or processing of dsRNAs, or inhibit the amplification, spreading, or stability of siRNAs. unc-47, a probable GABA transporter, is expressed within the 26 C. elegans GABAergic neurons (McIntire et al., Nature 389:870-6, 1997). Animals carrying an integrated unc-47::GFP (green fluorescent protein) fusion gene showed little or no decline in GFP fluorescence following feeding on bacteria expressing GFP dsRNA (FIGS. 1A and 1B). We screened ˜50,000 haploid genomes, following ethyl methanesulfonate mutagenesis, for mutants that exhibit a dramatic decrease in the number of neurons that express GFP following feeding on Escherichia coli that produce GFP dsRNA, compared to wild-type animals, but show a normal pattern of unc-47::GFP fluorescence when feeding on E. coli that do not express GFP dsRNA. As a secondary screen, candidate mutants were tested for increased sensitivity to dsRNAs derived from endogenous chromosomal loci (detailed below). Among the 19 candidate mutants isolated from this genetic screen, two of the strongest enhancers of RNAi define the gene enhanced RNAi-1 (eri-1). The Eri phenotypes of both eri-1 alleles, which are predicted to be null alleles, were indistinguishable and both alleles showed a temperature sensitive (ts) sterile phenotype.

eri-1(mg366) mutant animals displayed a pattern and intensity of unc-47::GFP fluorescence under normal growth conditions. eri-1(mg366) animals feeding on bacteria expressing GFP dsRNA exhibited a 70% decrease in the number of GABAergic neurons with GFP fluorescence (FIGS. 1C and 1D, Table 1).

TABLE 1
eri-1 animals show enhanced sensitivity to dsRNAs

| Genotype | dsRNA | Phenotype scored | Phenotype Percentage |
|---|---|---|---|
| unc-47::GFP | Control Vector | # neurons + GFP | 19.8 +/− 1.5 |
| eri-1; (mg366); unc- | Control Vector | # neurons + GFP | 19.4 +/− 1.5 |
| 47p::GFP | | | |
| unc-47::GFP | GFP dsRNA | # neurons + GFP | 18.5 +/− 2 |
| eri-1(mg366); unc-47p::GFP | GFP dsRNA | # neurons + GFP | 6.6 +/− 3 |
| rrf-3(pk1426); unc-47p::GFP | GFP dsRNA | # neurons + GFP | 6.1 +/− 2 |
| eri-1(mg366); unc- | GFP dsRNA | # neurons + GFP | 18.4 |
| 47p::GFP; | | | |
| T07A9.5 + daf-18 operon | | | |
| eri-1(mg366); unc- | GFP dsRNA | # neurons + GFP | 19.3 |
| 47p::GFP; sur-5p:: T07A9.5 | | | |
| N2 | lin-1 dsRNA | % multi-vul | 0.2 +/− 0.2 |
| eri-1(mg366) | lin-1 dsRNA | % multi-vul | 56 +/− 2.9 |
| rrf-3(pk1426) | lin-1 dsRNA | % multi-vul | 63 +/− 4.2 |
| eri-1(mg366); rrf-3(pk1426) | lin-1 dsRNA | % multi-vul | 57 +/− 2.2 |
| eri-1(mg366); rde-1(ne300) | lin-1 dsRNA | % multi-vul | 0 |
| eri-1(mg366); rde-4(ne299) | lin-1 dsRNA | % multi-vul | 0 |
| eri-1(mg366; mut-16 | lin-1 dsRNA | % multi-vul | 0 |
| eri-1(mg366); sid-1(qt3) | lin-1 dsRNA | % multi-vul | 0 |
| N2 | daf-2 dsRNA | % dauers | 0 |
| eri-1(mg366) | daf-2 dsRNA | % dauers | 35 +/− 5 |
| N2 | daf-19 dsRNA | % dauers | 0 |
| eri-1(mg366) | daf-19 dsRNA | % dauers | 12 +/− 5 |
| N2 | hmr-1 dsRNA | % lethality | 27 +/− 8 |
| eri-1(mg366) | hmr-1 dsRNA | % lethality | 100% |
| N2 | dpy-13 dsRNA | Dumpy | + |
| eri-1(mg366) | dpy-13 dsRNA | Dumpy | ++++ |
| N2 | unc-86 dsRNA | uncoordinated | − |
| eri-1(mg366) | unc-86 dsRNA | uncoordinated | + |

Interestingly, we observed an all or nothing response within individual neurons. GFP was either completely quenched by GFP dsRNA or was unaffected (FIG. 1). The enhanced RNAi phenotype of eri-1(mg366) was tested with a second GFP fusion gene, tub-1::GFP, which is expressed within the sensory neurons (FIGS. 2A-D). tub-1::GFP was not silenced in wild-type animals feeding on E. coli expressing GFP dsRNA, but eri-1(mg366); tub-1::GFP animals exhibited a 75% decrease in the number of neurons exhibiting GFP fluorescence following exposure to GFP dsRNA.

eri-1 Mutants Exhibited a Generalized Increase in Their Sensitivity to RNAi

eri-1(mg366) also sensitized animals to dsRNA targeted against endogenous chromosomal loci, some of which do not act only in neurons. For example, lin-1 mutations cause a multi-vulva (Muv) phenotype (Beitel et al., Genes Dev 9:3149-62, 1995), but for unknown reasons lin-1 is refractory to RNAi in wild-type animals. eri-1(mg366) animals fed E. coli expressing lin-1 dsRNA exhibited a Muv phenotype (Table 1). eri-1(mg366) animals also showed enhanced sensitivity to dpy-13, daf-19, myo-2, hmr-1, unc-86, and daf-2 dsRNAs compared to wild-type animals (Table 1). Two of these genes, unc-86 and daf-19, are expressed exclusively within neurons. The lin-1, dpy-13, myo-2, unc-22 and hmr-1 mRNAs are unlikely to be expressed in neurons. Therefore, although eri-1 was isolated in a screen for enhanced neuronal RNAi, loss of eri-1 activity caused a generalized increase in the efficacy of RNAi in most tissues.

While RNAi against many refractory target genes was successful, eri-1 mutations did not enhance RNAi in the nervous system to the point that feeding on E. coli expressing dsRNAs derived from the majority of unc genes was sufficient to induce the expected loss of function phenotypes. Feeding dsRNAs derived from the unc-13, unc-17, unc-25, and unc-47 loci did not produce the Unc phenotypes predicted by the loss of function mutations.

### T07A9.5 Encodes ERI-1

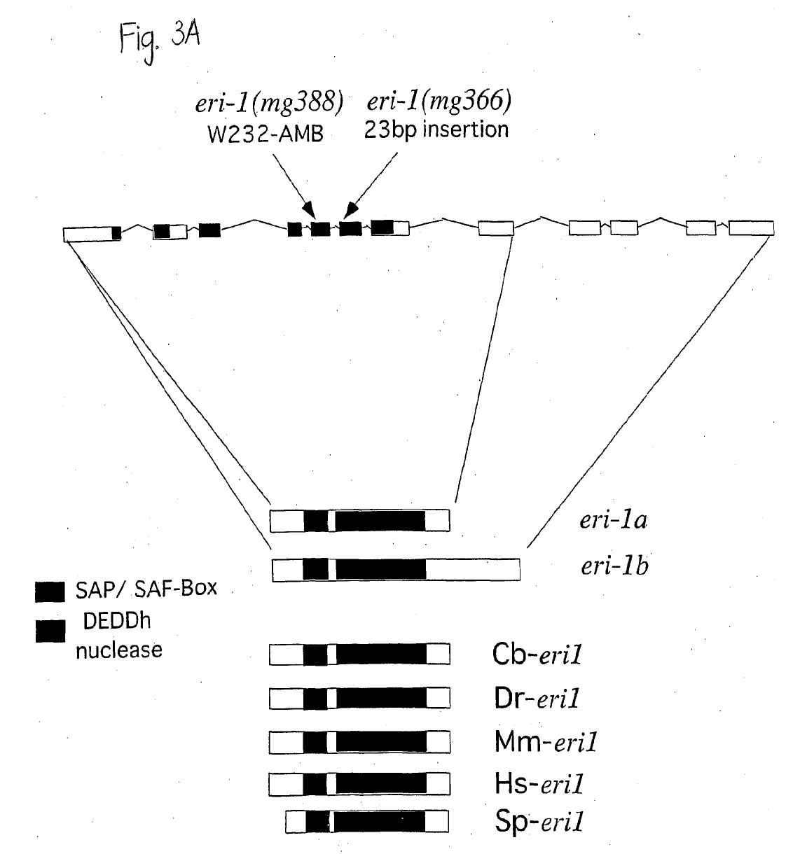

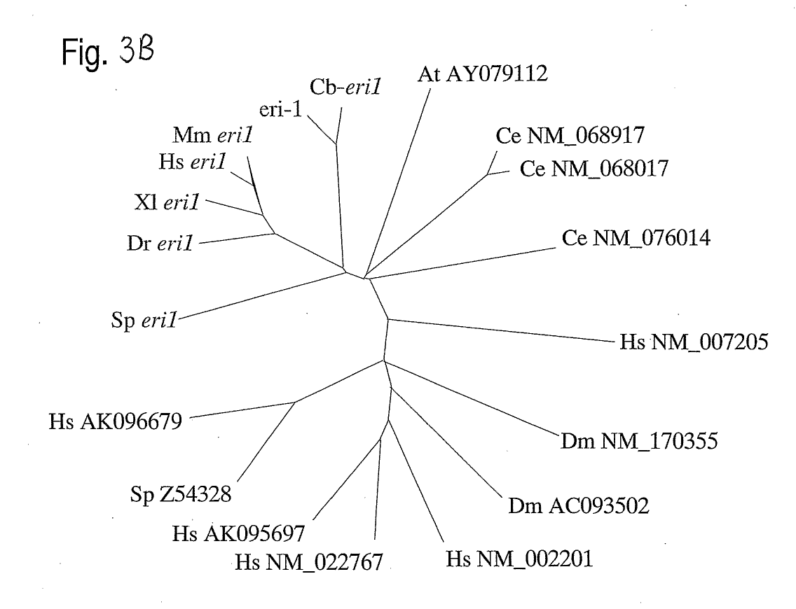

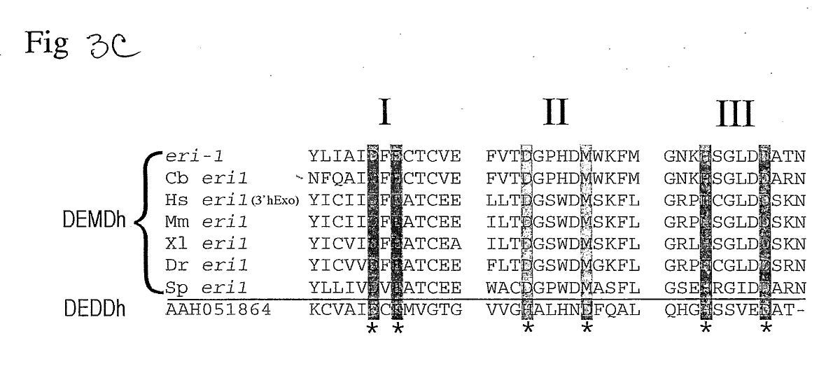

Genetic mapping localized eri-1 to a one map unit region on the far left arm of chromosome IV. Within this interval, we identified a 23 bp insertion within the open reading frame T07A9.5 in eri-1(mg366). This insertion encodes the addition of 7 amino acids followed by a premature stop codon (FIG. 3A). A second independently isolated allele, eri-1 (mg388), has a point mutation within T07A9.5 that specifies a stop codon in place of W231 (FIG. 3A). Both eri-1(mg366) and eri-1(mg388) are predicted to stop translation upstream of conserved domains (see below) and thus are likely to reveal the null phenotype: a viable but temperature sensitive sterile strain with enhanced sensitivity to RNAi.

T07A9.5 was predicted to reside downstream in an operon with the daf-18 gene. Transformation of DNA containing the predicted daf-18 and T07A9.5 operon (including 1.5 kb of upstream promoter sequence) into eri-1(mg366) animals rescued the ts sterility and enhanced RNAi phenotypes associated with eri-1(mg366) (Table 1). Transformation of T07A9.5 alone, including 5′ sequences up to the daf-18 locus, did not rescue eri-1(mg366), consistent with T07A9.5 being co-transcribed with daf-18. Transformation of T07A9.5 expressed under the control of the ubiquitously expressing heterologous sur-5 promoter (Gu et al., Mol Cell Biol 18: 4556-64 1998) rescued the enhanced RNAi and ts sterility of eri-1(mg366) (Table 1, and data not shown). Thus, we have shown that T07A9.5 corresponds to eri-1.

eri-1 Encodes a Polypeptide Containing a DEDDh-like 3′-5′ Exonuclease Domain

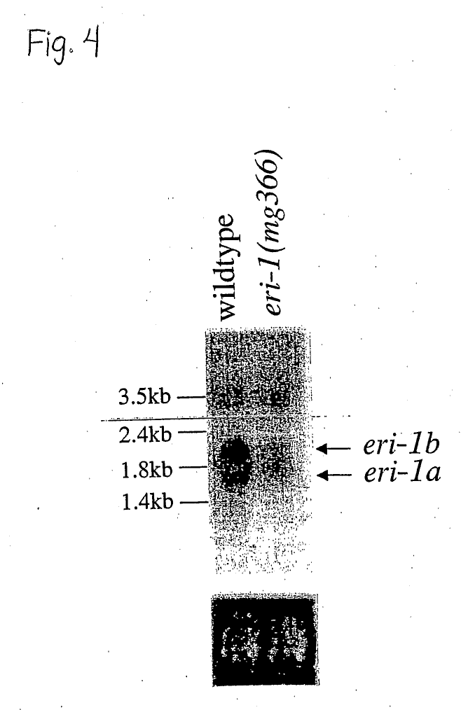

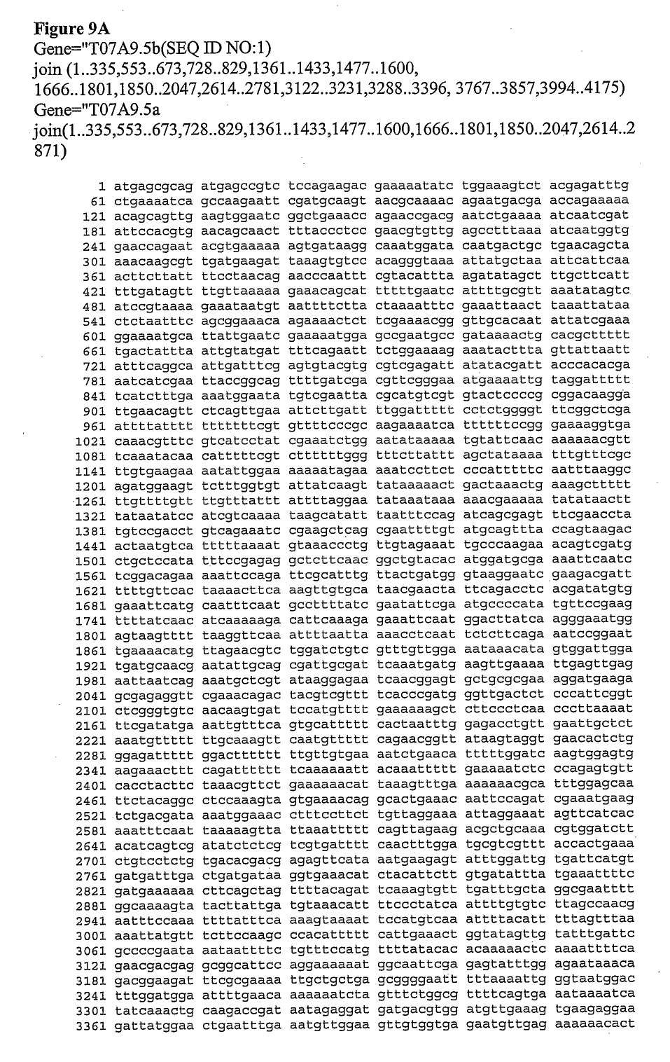

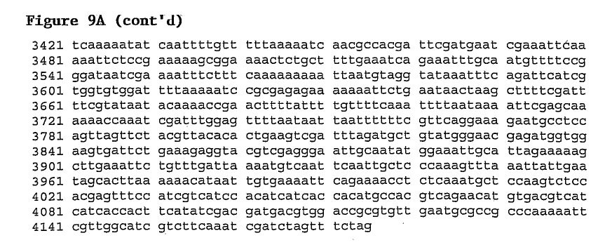

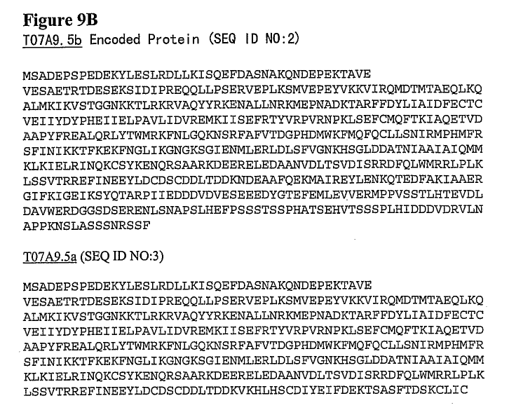

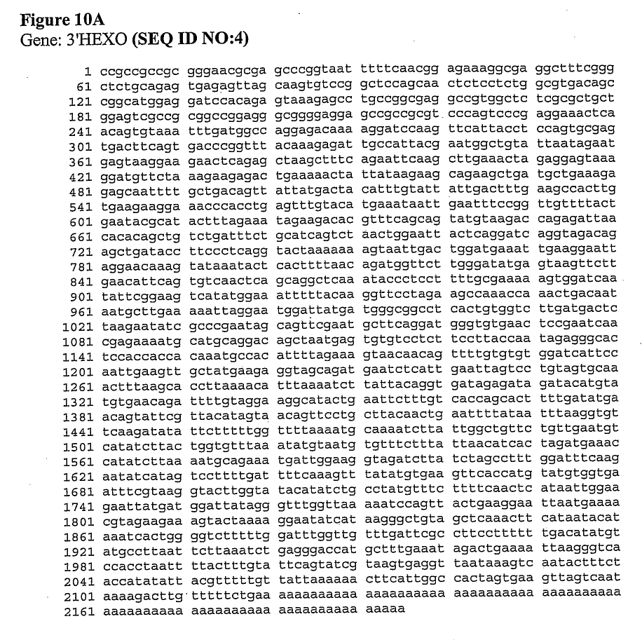

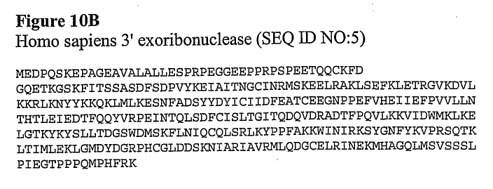

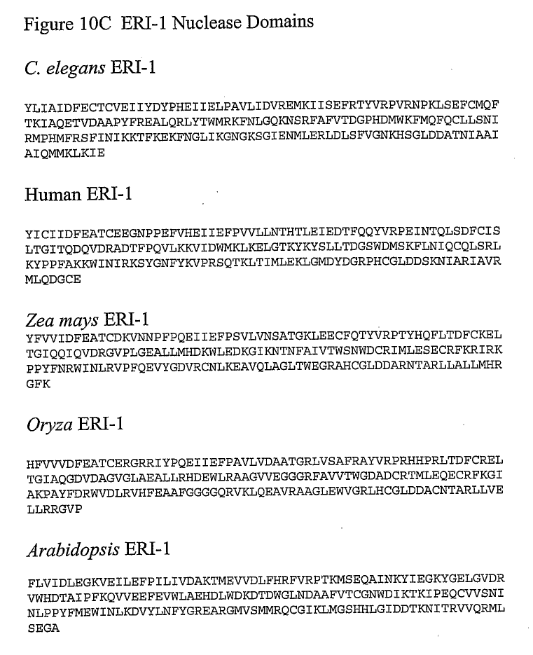

Northern blot analysis indicated that eri-1 encodes two equally abundant splice variants of approximately 1400 and 1800 nucleotides in length; termed eri-1a and eri-1b respectively (FIG. 4). Both splice variants of eri-1 encode a protein bearing a DEDDh-like 3′-5′ exonuclease domain and a Saf-A/B, Acinus, and PIAS (SAP, also termed SAF-BOX) domain (FIG. 3A). Members of the DEDDh family of nucleases include RNase T, oligoribonuclease, and the proofreading subunit of E. coli DNA polymerase III (Zuo et al., J Biol Chem 277:50155-9, 2002). The DEDDh family member RNAse T utilizes dsRNAs with 3′ overhangs as preferred substrates (Zuo et al., Nucleic Acids Res 29:1017-26, 2001). E. coli oligoribonuclease (orn) enzymes are required for the end degradation of mRNAs; orn mutants accumulate small 2-5 nucleotide mRNA degradation products (Ghosh et al., Proc Natl Acad Sci USA 96:4372-7, 1999). The putative human ortholog of eri-1 was recently biochemically purified as a factor that binds and degrades a short 3′ 4 bp overhang in a dsRNA stem loop structure at the 3′ terminus of a histone mRNA (Dominski et al., Mol Cell 12: 295-305, 2003). The nucleic acid and amino acid sequences of C. elegans and human eri-1 are shown in FIGS. 9 and 10, respectively. SAP/SAF-BOX domains show structural similarities to homeodomain DNA binding proteins, and one member of this family, SAF-A, has the ability to bind DNA (Kipp et al., Mol Cell Biol 20:7480-9, 2000). Although the SAP domain of ERI-1 suggests a possible function in the nucleus, the cytoplasmic localization of ERI-1 suggests that if ERI-1 has a nuclear function, it is transient. It is also possible that in the context of ERI-1 the SAP domain binds dsRNA to stabilize interactions between RNA and the nuclease domain.

Database searches revealed a single probable ERI-1 ortholog in several vertebrate species and in fission yeast that bear a SAP domain immediately N-terminal to a DEDDh nuclease domain (FIG. 3A). Phylogenetic analysis utilizing either the exonuclease domain, or the SAP domain, demonstrated that these genes are likely to be orthologs (FIG. 3B). While we detected a probable ortholog of eri-1 in Schizosaccharomyces pombe we failed to detect an ortholog in Saccharomyces cerevisiae, consistent with the RNAi machinery being present in S. pombe and absent in S. cerevisiae (Aravind, Proc Natl Acad Sci USA. 97:11319-24, 2000). The putative orthologs of eri-1 all contained a unique active site, including two substituted residues thought to be directly involved in catalysis in related exonucleases (Hamdan et al., Structure (Camb) 10:535-46, 2002) (FIG. 3C). Because the substituted residues are located within the active site, near where the RNA phosphodiester bond is broken by the nuclease, it is possible that ERI-1 and its orthologs have nucleic acid substrates that are related to RNAseTs and oligoribonucleases, but have distinctive features. ERI-1 represents the founding member of a sub-family of DEDDh exonucleases that we term the DEMDh subfamily. Nuclease domains for C. elegans, human, Arabidopsis, rice, and corn ERI-1 polypeptides are shown in FIG. 10C.

ERI-1 is an siRNase

In Drosophila the RNAse III enzyme Dicer cleaves dsRNAs into small 21-23 nucleotide ds siRNAs with short 2-4 nucleotide (nt) 3′ overhangs (Zamore et al., Cell 101:25-33, 2000). The 3′ overhangs of siRNAs are required for efficient siRNA mediated mRNA degradation (Elbashir et al., Embo J 20:6877-88, 2001). Injection of a synthetic 23 bp unc-22 siRNAs with 2 nucleotide 3′ overhangs caused at least fifty-five times more progeny of eri-1 (−) injected animals to exhibit an unc-22 loss of function phenotype than progeny of eri-1 (+) injected animals. Thus, we have demonstrated that ERI-1 functions downstream of the processing of trigger dsRNA into siRNAs (Caplen et al., Proc Natl Acad Sci USA 98:9742-7, 2001) (Table 2). Moreover the physiologic response to injected siRNAs was prolonged in eri-1 (−) animals compared to eri-1 (+) animals (Table 2).

TABLE 2
eri-1 mutant nematodes display enhanced sensitivity to dsRNAs
and synthetic Dicer products

| | Feeding dsRNA or | | Phenotype |
|---|---|---|---|
| Genotype | *Injected siRNA | Phenotype | Percentage |
| unc-47::GFP | Control Vector | # neurons + GFP | 19.8 +/− 1.5 |
| eri-1; (mg366); unc-47p::GFP | Control Vector | # neurons + GFP | 19.4 +/− 1.5 |
| unc-47::GFP | GFP dsRNA | # neurons + GFP | 18.5 +/− 2 |
| eri-1(mg366); unc-47p::GFP | GFP dsRNA | # neurons + GFP | 6.6 +/− 3 |
| rrf-3(pk1426); unc-47p::GFP | GFP dsRNA | # neurons + GFP | 6.1 +/− 2 |
| eri-1(mg366); unc-47p::GFP; | GFP dsRNA | # neurons + GFP | 18.4 |
| T07A9.5 + daf-18 operon | | | |
| eri-1(mg366); unc-47p::GFP; | GFP dsRNA | # neurons + GFP | 19.3 |
| sur-5p:: T07A9.5 | | | |
| N2 | lin-1 dsRNA | % multi-vul | 0.2 +/− 0.2 |
| eri-1(mg366) | lin-1 dsRNA | % multi-vul | 56 +/− 2.9 |
| rrf-3(mg373) | lin-1 dsRNA | % multi-vul | 63 +/− 4.2 |
| eri-1(mg366); rrf-3(pk1426) | lin-1 dsRNA | % multi-vul | 57 +/− 2.2 |
| eri-1(mg366); rde-1(ne300) | lin-1 dsRNA | % multi-vul | 0 |
| eri-1(mg366); rde-4(ne299) | lin-1 dsRNA | % multi-vul | 0 |
| eri-1(mg366; mut-16(ne322) | lin-1 dsRNA | % multi-vul | 0 |
| eri-1(mg366); sid-1(qt2) | lin-1 dsRNA | % multi-vul | 0 |
| N2 | daf-19 dsRNA | % dauers | 0 |
| eri-1(mg366) | daf-19 dsRNA | % dauers | 12 +/− 5 |
| N2 | hmr-1 dsRNA | % lethality | 13 +/− 15 |
| eri-1(mg366) | hmr-1 dsRNA | % lethality | 99 +/− 2 |
| rrf-1(pk1417) | hmr-1 dsRNA | % lethality | 9 +/− 21 |
| eri-1(mg366); rrf-1(pk1417) | hmr-1 dsRNA | % lethality | 99 +/− 0.3 |
| N2 | dpy-13 dsRNA | Dumpy | + |
| eri-1(mg366) | dpy-13 dsRNA | Dumpy | ++++ |
| N2 | unc-86 dsRNA | uncoordinated | − |
| eri-1(mg366) | unc-86 dsRNA | uncoordinated | + |
| N2 | *unc-22 25 bp siRNA | twitcher, day 1 | 16 +/− 2 |
| eri-1(mg366) | *unc-22 25 bp siRNA | twitcher, day 1 | 67 +/− 7 |
| N2 | *unc-22 25 bp siRNA | twitcher, day 2 | 1 +/− 1 |
| eri-1(mg366) | *unc-22 25 bp siRNA | twitcher, day 2 | 55 +/− 12 |

Table 2 Legend:
L4 nematodes of the indicated genotype were placed on HT115 bacteria containing control vector or expressing dsRNA derived from the indicated gene (dsRNA). For GFP RNAi experiments, L4 progeny were scored for GFP fluorescence in ventral cord neurons. dsRNAi expressing bacteria were obtained from the Ahringer library³. For lin-1 RNAi experiments, adult animals were scored positive if they exhibited more than one vulva. For dpy-13 RNAi experiments + indicates weak dumpy phenotype and ++++ indicates very strong dumpy phenotype. For hmr-1 the % of eggs that that survived to adulthood is shown. For unc-86 RNAi experiments − indicates no Unc phenotype and + indicates strong Unc phenotype. +/− indicates standard errors of at least three independent experiments. unc-22 siRNAs were micro-injected 3 independent times and L3-L4 progeny from sequential egglays (day 1-2) were scored¹⁸.

Injected unc-22 siRNAs were more abundant in the progeny of eri-1 (−) animals than in the progeny of eri-1 (+) animals (FIG. 6A). Similarly, progeny of eri-1 (−) animals fed bacteria expressing dsRNA from the pos-1 gene accumulated more pos-1 siRNAs than the progeny of eri-1 (+) animals (FIG. 6B). In vitro, C. elegans ERI-1 and the probable human ERI-1 ortholog partially degraded a synthetic Dicer product; a double stranded siRNA with 2 nucleotide 3′ overhangs, but failed to degrade single stranded siRNAs or a single stranded siRNA hybridized to a 220 nucleotide unc-22 message (FIG. 6C). The increased sensitivity to siRNAs and increased abundance and stability of primary siRNAs in eri-1 animals, and the biochemical activity of ERI-1, indicated that ERI-1 is an siRNAse that inhibits RNAi by degrading the 3′ overhangs of Dicer products. siRNAs lacking 3′ overhangs may be non-functional because they fail to enter the RNAi induced silencing complex (RISC). Alternatively, 3′ resected siRNAs generated by ERI-1 in vitro may be unstable in vivo; they are not observed in eri-1 (+) animals following injection of siRNAs (FIG. 6A). In vivo, additional nucleases, or possibly ERI-1 in conjunction with a RNA helicase, may catalyze the complete degradation of siRNAs.

eri-1 Functions Downstream of the Processing of Trigger dsRNA into siRNAs.

To determine where in the RNAi pathway eri-1 functions, genetic epistasis analysis was performed between eri-1 and mutants defective in normal RNAi responses. We constructed double mutant combinations containing eri-1 and five genes required for RNAi; the RNAi defective argonaute-like rde-1, the dsRNA binding protein rde-4, the RNA dependent RNA polymerase rrf-1, the RNAi defective mutator gene mut-16, and the systemic RNAi defective mutant sid-1^{19 20 21 22}. rde-1, rde-4, mut-16, and sid-1 mutations were epistatic to eri-1 for sensitivity to all dsRNAs tested (Table 1). In contrast, eri-1 was epistatic to rrf-1 for sensitivity to hmr-1 dsRNA suggesting that amplification of secondary siRNAs in somatic tissues is not essential for eri-1 to enhance RNAi (Table 1) (Sijen, et al., Cell 107:465-76, 2001). Thus, consistent with the biochemical activity of ERI-1, the increased RNAi sensitivity of eri-1 animals likely depends on the production of primary siRNAs by the canonical RNAi pathway.

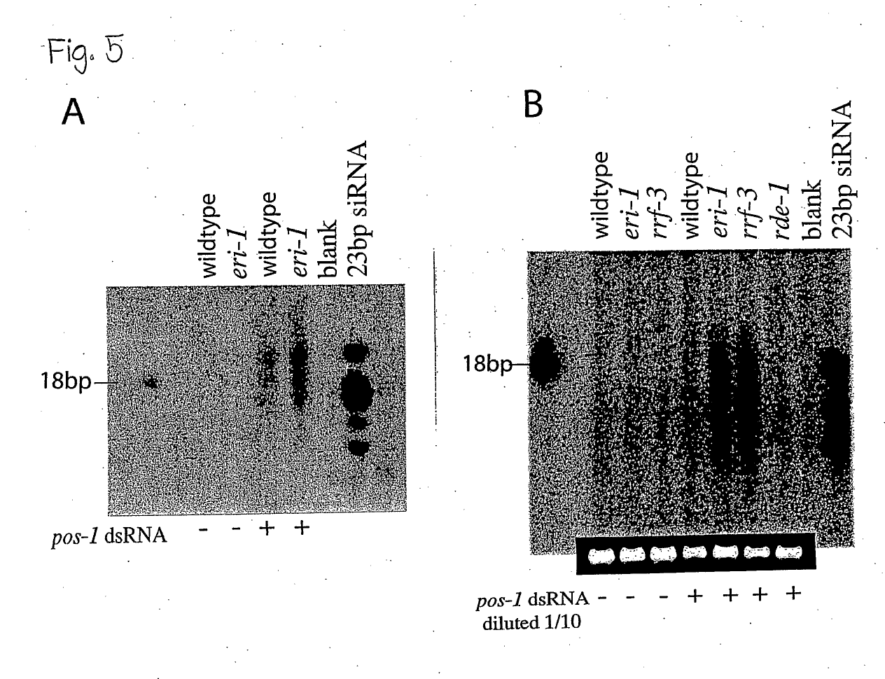

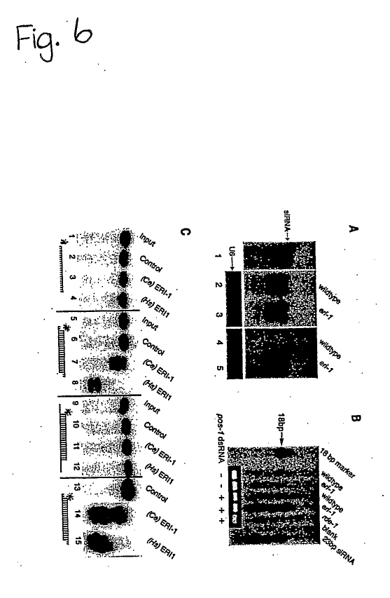

The enhanced RNAi phenotype of eri-1 could be due to increased feeding or increased uptake of dsRNA from bacteria expressing dsRNA, increased processing of dsRNA to siRNAs, increased half life of siRNAs, or a more effective mRNA degradation response to a given amount of siRNA. Injection of lin-1 dsRNA directly into animals produced 4% Muv animals while injection of lin-1 dsRNA into eri-1 animals produced 54% Muv animals, demonstrating that eri-1 enhances RNAi downstream of feeding and the uptake of dsRNA. Injection of synthetic double stranded 25 bp unc-22 siRNAs with 2 bp 3′ overhangs increased the number of progeny that exhibited an unc-22 phenotype in eri-1 animals by a factor of ten relative to the number of unc progeny produced when wild-type worms are similarly treated (Table 1). This demonstrated that ERI-1 functions downstream of the processing of trigger dsRNA into siRNAs. In addition, injected unc-22 siRNAs were more abundant in the progeny of eri-1 animals when compared to the progeny of injected wild-type animals (FIG. 5A). Similarly, progeny of eri-1(mg366) animals fed bacteria expressing dsRNA from the pos-1 gene accumulated more pos-1 siRNAs than the progeny of wild-type animals (FIG. 5B).

eri-1 Expression Pattern

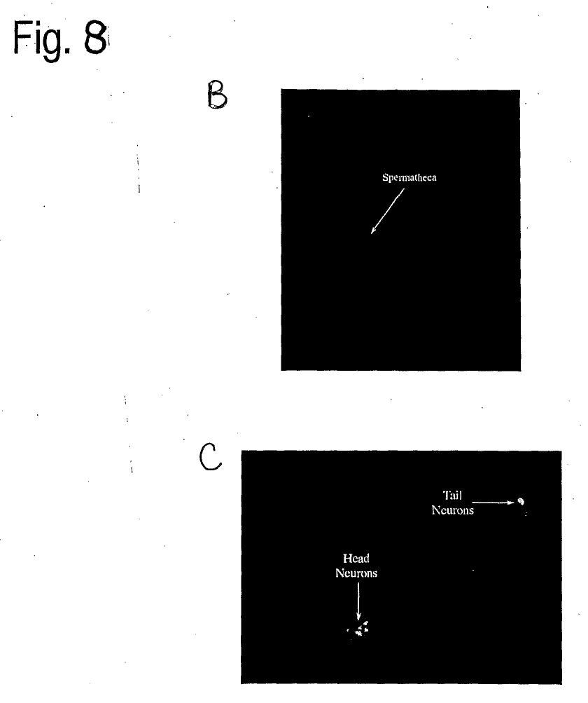

To determine in which tissues and sub-cellular compartment ERI-1 functions, we generated a fusion gene between GFP and the predicted eri-1 promoter, termed eri-1P::GFP. We also fused GFP to the full-length ERI-1b protein; termed eri-1b::GFP. Both fusion constructs contained the daf-18 genomic region and 1.5 kb of upstream promoter. eri-1 mutant animals stably expressing eri-1b::GFP were rescued for both enhanced RNAi and ts sterility, indicating that this fusion gene is functional and representative of endogenous eri-1 expression. In three independent lines we observed GFP expression within the developing somatic gonad and a subset of neurons (FIG. 6). In adult animals ERI-1 was expressed in neurons and gonadal expression was restricted to the spermatheca (FIG. 8). Within neurons ERI-1 was predominantly localized to the cytoplasm (FIG. 6). The eri-1P::GFP promoter fusion, which was expected to reveal the pattern of expression of both eri-1a and eri-1b, showed a similar pattern of expression pattern to the eri-1b::GFP. This fusion protein, which contained only the first five residues of ERI-1 was not localized preferentially to the cytoplasm. With this construct we also observed a low level of ubiquitous expression throughout the animal. The high level expression of ERI-1 in a subset of neurons may, at least in part, explain the relative inefficiency of RNAi within these neurons in wild-type animals. The low level of ubiquitous expression revealed by eri-1P::GFP may explain the observed generalized increase in the efficacy of RNAi observed in eri-1 animals.

The ERI-1::GFP sub-cellular localization and the expression pattern or intensity did not change following exposure to dpy-13, lin-1, daf-2, or unc-11 dsRNAs, suggesting that expression of the ERI-1 nuclease was not induced by dsRNA exposure. It is tantalizing that eri-1 is located in an operon with daf-18, the worm ortholog of PTEN, which acts in the insulin-like signalling pathway that regulates aging and stress responses. This coupling of eri-1 and a stress responsive pathway suggests that the function of eri-1, and in turn, the intensity of RNAi, may be coupled to stress inputs, consistent with theories that RNAi is a form of pathogen resistance.

ERI-1 Nuclease-Resistant siRNAs

C. elegans eri-1a and human eri-1 cDNAs were appended with a C-terminal T7 promoter and an N-terminal FLAG epitope and transcribed in vitro. These mRNAs were then translated in reticulocyte lysates and the resulting ERI-1 fusion proteins were immunoprecipitated with α-FLAG conjugated agarose beads and eluted with a FLAG peptide. In vitro translated and immunoprecipitated ERI-1 and human ERI-1 or mock translation (control) were incubated as described in Dominski et al., (Mol Cell 12: 295-305, 2003) with ds unc-22 siRNAs containing P³² 5′ end labelled unc-22 sense oligonucleotides, which contained five adenosine (A), uracil (U), cytosine (C), or guanosine (G) bases at their 3′ terminus. As shown in FIG. 11, while siRNAs having five 3′ adenosine (A), uracil bases were susceptible to nuclease degradation, siRNAs having five cytosines or guanosines were resistant to ERI-1 nuclease activity.

eri-1 RNAi of Dpy-13

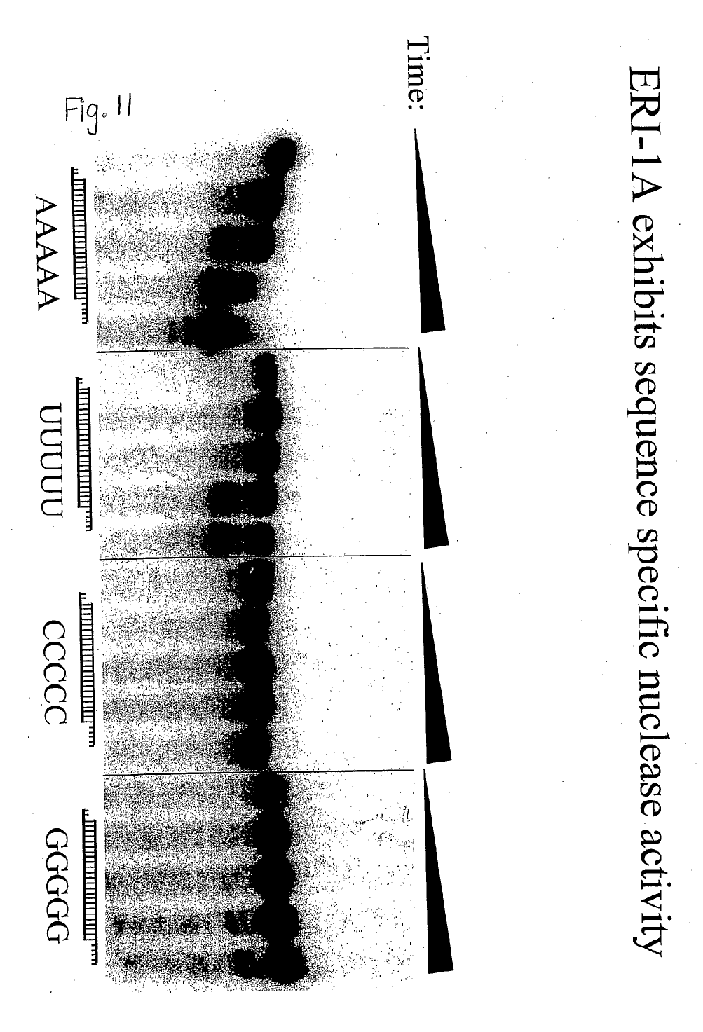

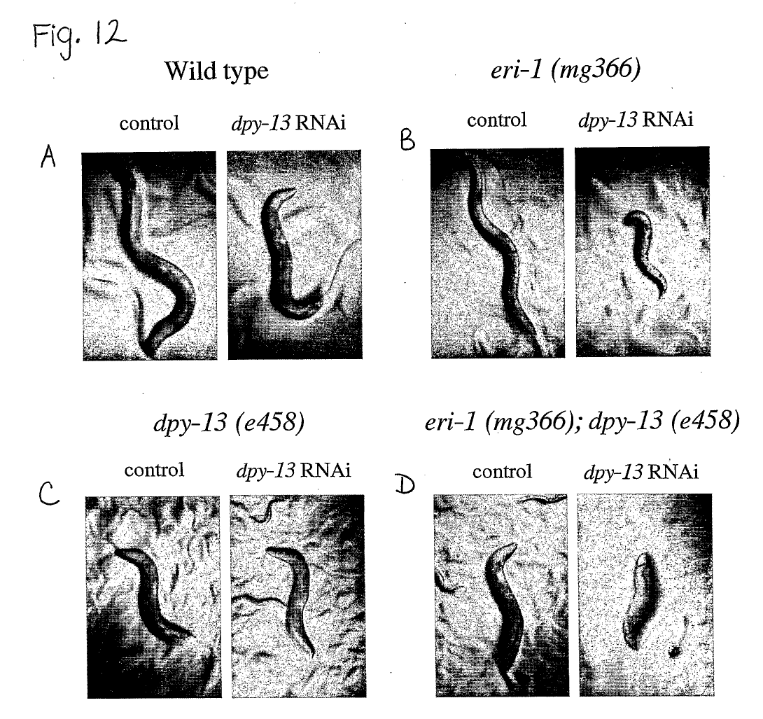

Wild-type nematodes fed bacteria expressing dsRNA targeting the dpy-13 collagen gene display a very subtle Dpy phenotype (FIG. 12A). Under similar conditions eri-1 and rrf-3 mutant nematodes display a Dpy phenotype that is more dramatic than that displayed by nematodes that are homozygous for a dpy-13 null allele (FIGS. 12B and 12C). This phenotype does not increase in severity when eri-1(mg366); dpy-13(e458) double mutant nematodes are fed dpy-13 dsRNA.

Given that dpy-13 encodes a collagen gene that is highly homologous at the DNA and RNA level to other C. elegans collagen genes, and without being tied to any particular theory, it is likely that, in an eri-1 or rrf-3 mutant, dpy-13 RNAi targets not only dpy-13, but other homologous collagen genes as well. Such a theory could account for the more dramatic Dpy phenotype observed in eri-1 mutant nematodes (FIGS. 12B and 12D).

Thus, drugs that inhibit ERI-1 nuclease activity could increase the tolerance of RNAi for siRNA/target sequence mismatches, and allow a single siRNA to target multiple members of a single multigene family or rapidly evolving viruses, where the siRNA fails to complement the target sequence at one or more nucleotides. Under circumstances where it is desirable to maximize RNAi mismatch tolerance, siRNAs are designed to target sequences that are highly conserved among members of a gene family.

Alternatively, to maximize specificity, siRNAs are designed to target highly divergent sequences within the genome of an organism. “Highly divergent sequences” are those that do not have significant homology with any other genomic sequences. siRNAs that target highly divergent sequences are likely to silence only the target gene.

eri-1 likely Functions in a Genetic Pathway with rrf-3

eri-1 and rrf-3 are likely to function in the same genetic pathway. The C. elegans genome encodes four RNA dependent RNA polymerases (RdRPs). Two of these RdRPs are required for RNAi and are thought to function in the amplification of secondary siRNAs (Sijen et al., Cell 107:465-76, 2001). Paradoxically inactivation of one of the other two RdRPs, rrf-3, results in an enhanced RNAi phenotype, suggesting that this RdRP inhibits the amplification of siRNAs by either antagonizing the other RdRPs or by shunting siRNAs along a distinct pathway. The genetic screen from which the eri-1 alleles emerged also identified an allele of rrf-3 (Sijen, et al., Cell 107:465-76, 2001; Simmer et al., Curr Biol 12:1317-9, 2002). rrf-3(mg373) is a G817E point mutation in an evolutionarily invariant glycine residue within the RdRP domain of RRF-3. rrf-3(mg373) exhibited as severe an enhanced RNAi phenotype as the deletion allele rrf-3(pk1426) suggesting that G817 plays an essential role in RRF-3. eri-1(mg366) and rrf-3(mg373) mutant animals showed an equivalent hypersensitivity to RNAi (Table 1) and both mutants caused an increase in the levels of siRNAs following RNAi induction (FIG. 5B). eri-1 mutant animals also shared several pleiotropic phenotypes with rrf-3 mutants; increased chromosome nondysjunction, as measured by the production of XO males, and temperature sensitive sterility. Both eri-1 and rrf-3 mutations showed a transgene silencing phenotype; they were both capable of silencing a rol-6 transgene (Simmer et al., Curr Biol 12:1317-9, 2002). The eri-1(mg366); rrf-3(pk1426) double mutant did not exhibit additional enhanced RNAi phenotypes or synthetic developmental phenotypes compared to the single mutant animals (Table 1). The shared molecular and pleiotropic phenotypes, and the lack of any additional RNAi sensitivity in the double mutant animal, indicated that eri-1 and rrf-3 are likely to function in the same genetic pathway.

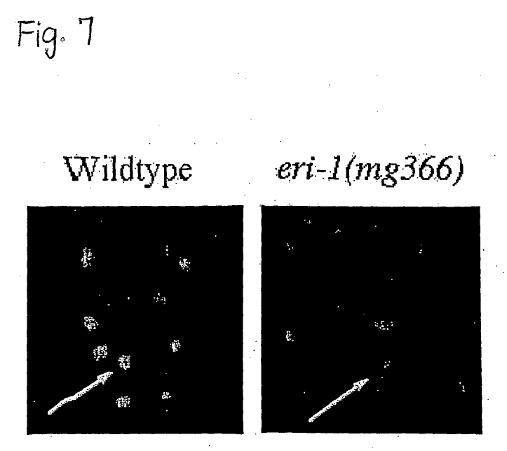

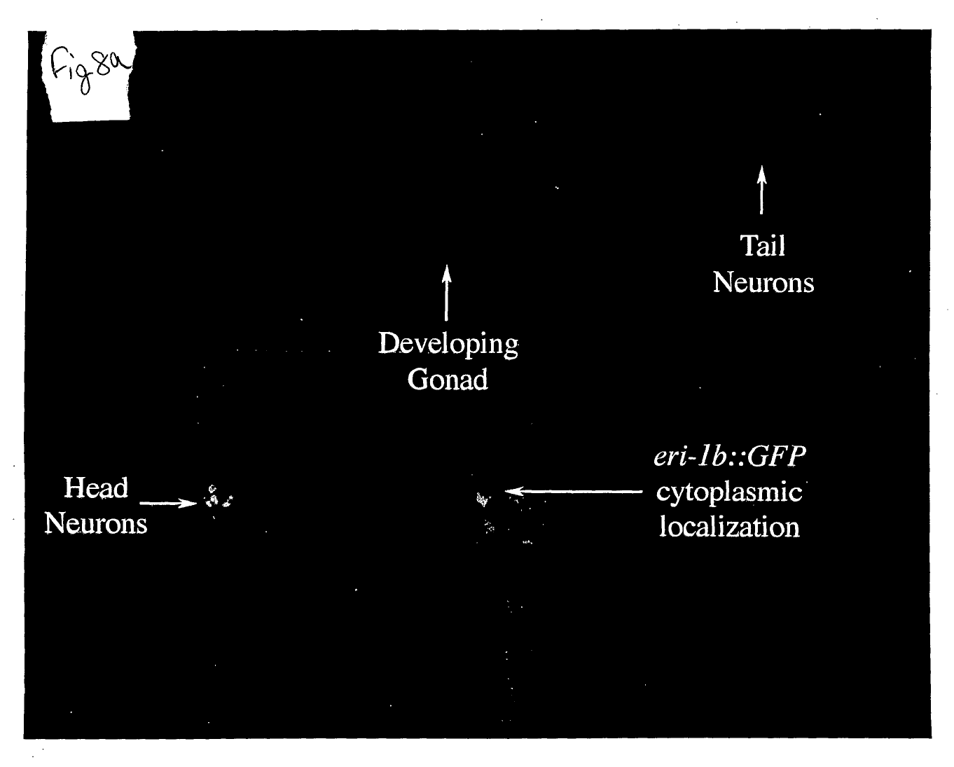

The temperature sensitive (ts) sterility of eri-1 null mutant animals is due to defective sperm development; the sterility was rescued by mating to wild-type males or eri-1 males grown at the permissive temperature. DAPI staining of eri-1 gonads revealed a normal mitotic expansion of the germline, but sperm nuclei exhibited gross morphological defects (FIG. 7). rde-1, rde-4, and sid-1 failed to suppress the sperm defect in eri-1 animals. It is conceivable that the sperm defect present in eri-1 mutant worms is due to improper regulation of histone (or other) mRNAs. If this is the case, however, it is difficult to imagine why loss of the RdRP rrf-3 would also result in this specific phenotype. miRNAs are short ˜23 bp RNAs that share processing machinery, such as Dicer and Argonaute genes, with the RNAi pathway (Reinhart et al., Nature 403:901-6, 2000; Grishok, Science 287:2494-7, 2001; Ketting et al. Nature 404:296-8, 2000). Loss of ERI-1 or RRF-3 may induce misregulation of endogenous miRNAs normally required for proper spermatogenesis. Alternatively, an endogenous RNAi pathway, not dependent on rde-1, rde-4, and sid-1, may be required for spermatogenesis in C. elegans.

The molecular basis of how ERI-1 and RRF-3 work in a pathway to inhibit RNAi is unclear. It is likely that the ERI-1 exonuclease degrades siRNAs to limit an episode of RNAi. Our molecular data demonstrated that both eri-1 and rrf-3 loss of function mutations caused an accumulation of siRNAs, suggesting that the RRF-3 RdRP acts to inhibit the production or half-life of siRNAs. It is possible that RRF-3 inhibits the generation of siRNAs and ERI-1 inhibits their stability. Alternatively, ERI-1 and RRF-3 could work together to shunt siRNAs into another pathway not directly mediating RNAi.

The identification of the eri loci indicated that the RNAi machinery was under substantial negative regulation. This negative regulation may function to limit the intensity of an episode of RNAi or inhibit RNAi in particular cell types. The eri-1 orthologs in mammals and fungi may also inhibit RNAi/PTGS so that inhibition of their activity by drugs that target the unique active site of DEMDh nucleases may allow more efficient RNAi. Inhibition of the eri-1 orthologs, for example by drugs that specifically target the unique active site of this nuclease, may allow for the more efficient use of RNAi in a wide variety of clinical therapies.

The above-described experiments were carried out as follows.

### Strains

The following strains were used in the above-described experiments: EG1285; lin-15(n765ts); oxls12 (unc-47::GFP) (McIntire et al., Nature 389:870-6, 1997); NL2099; rrf-3(pk1426) (Sijen, et al., Cell 107:465-76, 2001); GR1373: eri-1(mg366); GR1374: eri-1(mg366), lin-15(n765ts);oxls12(unc-47::GFP); GR1375: eri-1(mg388); lin-15(n765ts);oxls12(unc-47::GFP); GR1377: rrf-3(pk1426); lin-15(n765ts);oxls12(unc-47::GFP); GR1378: eri-1(mg366); lin-15(n765ts);oxls12(unc-47::GFP);T07A9.5+daf-18operon; GR1376: eri-1(mg366);tub-1::GFP; GR1386: rrf-3(pk1426);tub-1::GFP; GR1379: eri-1(mg366);rrf-3(pk1426); GR1380: eri-1(mg366);rde-1(ne300); GR1381: eri-1(mg366);rde-1(ne300); GR1382: eri-1(mg366);rde-4(ne299); GR1383: eri-1(mg366);rde-4(ne299); GR1384: eri-1(mg366);mut-16(ne322)unc-13; GR1385: eri-1(mg366); sid-1(qt2);

Genetic Mapping of eri-1

eri-1(mg366) was mapped using a Hawaiian isolate of C. elegans (CB4856). 141 F2 recombinants were scored for eri-1 based upon sensitivity to GFP dsRNA, sensitivity to dsRNA of hmr-1, and/or the ts sterility phenotype. Single nucleotide polymorphism mapping established a right boundary on chromosome IV at position 1932 of cosmid C05G6, corresponding to a genetic map position of approximately −23.1. A two factor cross with dpy-9 (−27.3) yielded 11/190 recombinant chromosomes for a genetic distance of 2.8 map units. These mapping data placed eri-1 at approximately −24.5 on the left arm of LG IV.

### Transgenes

DNA for rescue of eri-1 was generated by pooling six independent PCR reactions from N2 animals. Amplified DNA corresponding to cosmid T07A9 positions 43255 to 32125 is termed T07A9.5+daf-18 operon. DNA was injected into eri-1(mg366) at 5 ng/ul with 20 ng/ul of tub-1::GFP marker DNA. Control lines were generated by injecting eri-1(mg366) animals with 20 ng/ul of tub-1::GFP marker DNA. Control lines did not rescue eri-1 phenotypes.

The sur-5p::eri-1 transgene was constructed by PCR fusion of 3.1 kb of the sur-5 promoter with full length genomic T07A9.5. The fusion was made at the predicted ATG of T07A9.5 and included the entire genomic sequence of T07A9.5 and 328 bp of 3′ sequence (to T07A9 position 32415). eri-1(mg366) animals were co-injected with 5 ng/ul of sur-5p::eri-1 DNA and 20 ng/ul of tub-1::GFP marker DNA. Control eri-1(mg366) lines were generated by injecting with 20 ng/ul of tub-1::GFP marker DNA. Control lines did not rescue eri-1 phenotypes.

The eri-1::GFP fusion construct was generated by two rounds of PCR fusion. T07A9 (position 43293 to 32746) was fused at the predicted stop codon of T07A9.5 to the ATG of PCR amplified GFP from construct pPD95.77 (provided by Andy Fire). This GFP does not contain any localization signals. The native 3′ UTR of T07A9.5 (T07A9 position 32116-32743) was then PCR fused to this DNA to generate a full length T07A9.5, fused to GFP, within the context of the native operon. This construct was injected into eri-1(mg366) animals at a concentration of 5 ng/μl. eri-1p::GFP fusion construct was also generated by PCR fusion. T07A9 (position 43293 to 36901) was fused to GFP amplified from pPD95.77. This fusion gene contains 1045 nucleotides of the unc-54 3′ UTR. eri-1p::GFP was injected into wild-type animals at 5 ng/ul.

### RNAi Assays

For RNAi assays L1 and L2 animals were fed on E. coli expressing dsRNA taken from the Ahringer RNAi library (Kamath et al., Nature 421:231-7, 2003) and grown at 20°. F1 progeny were scored for the indicated phenotypes. For injection experiments L4 animals (wild-type or eri-1(mg366) were injected with 2 μg/μl of lin-1 dsRNA. F1 progeny were scored as positive if they had more than one vulva.

### Inhibitory Nucleobase Oligomers

Inhibitory nucleobase oligomers (e.g., double stranded RNA (dsRNA), short interfering RNA (siRNA), antisense RNA, short hairpin RNA (shRNA), and mimetics thereof) decrease the expression of target genes. Using the nucleic acid sequence of eri-1, rrf-3(mg373), or plant or mammalian orthologs thereof, inhibitory oligonucleotides (e.g., nucleic acids or nucleobase oligomers) targeting eri-1 or rrf-3 genes may be identified. Inhibitory oligonucleotides targeting eri-1 or rrf-3 are useful for a variety of applications, including RNAi therapies

siRNA

Short twenty-one to twenty-five nucleotide double stranded RNAs effectively down-regulate gene expression in vitro, for example, in mammalian tissue culture cell lines (Elbashir et al., Nature 411:494-498, 2001) and in vivo (McCaffrey et al., Nature 418:38-9, 2002).

siRNAs also effectively downregulate viral gene expression in cultured cells. For example, siRNAs effectively inhibit gene transcription in HIV-1 (Coburn et al. J. Virol. 76:9225-31, 2002); respiratory syncytial virus (Bitko et al. BMC Microbiol. 1:34, 2001), and Influenza A virus (Ge et al., Proc Natl Acad Sci USA. 100:2718-23, 2003), and dsRNAs administered to cultured cells prevent infection of cultured cells with polio virus (Gitlin et al., Nature 418:430-4, 2002).

siRNAs and antisense oligonucleotides effectively downregulate viral gene expression in vivo. For example, RNA interference effectively targets a sequence from hepatitis C virus in vivo (U.S. Patent Application Publication 20030153519, McCaffrey et al., Nature 418:38-39, 2002; McCaffrey et al., Hepatology. 38:503-8, 2003).

Provided with the sequence of human eri-1 or rrf-3, siRNAs may be designed that enhance RNAi of target genes. Methods for designing siRNAs are known to the skilled artisan. (See, for example, Dykxhoorn Nature Rev Mol Cell Biol 4:457-467, 2003; Paddison et al. Genes Dev. 16:948-958, 2002; Paddison et al., Proc Natl Acad Sci USA. 99:1443-1448, 2002; Sohail et al., Nucleic Acids Res. 31:e38, 2003; Yu et al., Proc Natl Acad Sci USA. 99:6047-6052, 2002.) While various parameters are used to identify promising RNAi targets, the most effective siRNA and shRNA candidate sequences are identified by empirical testing.

In one example, human siRNAs are identified as follows. An eri-1 siRNA and an siRNA targeting a gene of interest are transferred into mammalian cells in culture. The administration of the eri-1 siRNA may be prior to, co-incident with, or shortly after the administration of an siRNA targeting a gene of interest. The expression of the gene of interest is compared in cells contacted with an eri-1 siRNA and in corresponding control cells not contacted with an eri-1 siRNA. siRNAs that decrease expression of a gene of interest in an eri-1 contacted cell relative to a control cell are useful in the methods of the invention.

Specific eri-1 siRNAs that enhance RNAi in vitro can be used in vivo as therapeutics and are especially useful in enhancing the inactivation of genes thought to be refractory to RNAi.

Improved Methods for Identifying siRNAs

Given that eri-1 encodes an siRNAse that inhibits RNAi, siRNAs that are resistant to the nuclease activity of ERI-1 are expected to have increased gene silencing activity. Such siRNAs can be designed or selected using the methods of the invention. For example, siRNAs having at least 2, 3, 4, or 5 3′ terminal cytosines or guanosines were resistant to ERI-1 nuclease activity.

Alternatively, siRNAs can be selected using screens to identify siRNAs that are resistant to ERI-1 nuclease activity. In one embodiment, a random siRNA library is screened to identify those siRNA sequences that are resistant to ERI-1 nuclease activity. For example, siRNAs are exposed to an ERI-1 polypeptide under conditions that allow ERI-1 susceptible siRNAs to be degraded. siRNAs that resist ERI-1 degradation are then isolated and characterized to determine the sequences that render them resistant.

### Therapeutic Uses of RNAi

eri-1 inhibitory nucleic acids are useful in enhancing therapeutic RNAi for the treatment of virtually any condition that requires gene silencing. For example, an eri-1 inhibitory nucleic acid enhances RNAi when administered in combination with an inhibitory nucleic acid that targets a gene that is expressed in a pathogen, in a neoplastic or hyperproliferating cell, in a genetic disorder, or in any disorder characterized by the expression of at least one mutant allele. Thus, eri-1 inhibitory nucleic acid compositions are useful for enhancing the treatment of a variety of pathological conditions, including but not limited to the treatment of pathogen infections (bacterial, viral, parasitic), hyperproliferative disorders (e.g., neoplasms, such as cancer), and genetic disorders resulting from the expression or overexpression of a gene or mutant allele. In addition, eri-1 inhibitory nucleic acids enhance RNAi that targets genes previously thought to be refractory to RNAi

For some applications, eri-1 is administered in combination with an inhibitory nucleic acid that targets a portion of a pathogen genome, where inactivation of a portion of a pathogen genome is sufficient to prevent, ameliorate, or eliminate infection by the pathogen. By enhancing RNAi of a pathogen genome, eri-1 compositions of the invention facilitate both the treatment and prevention of pathogen infections in a subject. Methods for the use of RNAi in the treatment of a pathogen infections are described, for example, in U.S. Patent Publications 20030219407, 20030203868, 20030206887, 2003020386, and 2003020386.

In other embodiments, eri-1 is administered in combination with an inhibitory nucleic acid that targets an endogenous gene of interest whose expression or overexpression induces a disease or disorder, such as a neoplasm. In one example, eri-1 is administered in combination with an inhibitory nucleic acid that targets a gene whose expression contributes to cancer. In other examples, eri-1 enhances RNAi of a targeted gene that promotes abnormal angiogenesis. In still other examples, eri-1 enhances RNAi used to treat a genetic disorder (e.g., familial hypercholesteremia, dominant forms of retinal degeneration, Parkinson's disease, spinobulbar muscular atrophy Huntington's disease, myotonic dystrophy, or other trinucleotide repeat disorders. Therapeutic RNAi methods that target endogenous genes are known to the skilled artisan. See, for example, U.S. Patent Publications 20030148519, 20030148519, 20030143204. In still other examples, eri-1 enhances allele specific RNAi, which allows allele-specific silencing of a mutant target allele, while not interfering with the expression of a wild-type allele. Such methods are described, for example, in Xia et al., (Nucleic Acids Res. 2003 Sep. 1; 31(17): e100), Abdelgany et al (Hum Mol Genet. 2003 Oct. 15; 12(20): 2637-44), and Caplen et al., (Human Molecular Genetics, 2002, Vol. 11, No. 2 175-184).

Methods for Producing siRNAs and Other Oligonucleotides

Methods for producing eri-1 siRNAs and other eri-1 inhibitory nucleobase oligomers are standard in the art. For example, an eri-1 siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. Proc Natl Acad Sci USA 98:9742-9747, 2001; Elbashir, et al. EMBO J 20:6877-88, 2001).

21-23 nucleotide eri-1 dsRNAs can be chemically synthesized by any method known to one of skill in the art, for example, using Expedite RNA phosphoramidites and thymidine phosphoramidite (PROLIGO, Boulder, Colo.). Synthetic oligonucleotides can be deprotected and gel-purified. dsRNA annealing can be carried out by any method known in the art, for example: a phenol-chloroform extraction, followed by mixing equimolar concentrations of sense and antisense RNA (50 nM to 10 mM, depending on the length and amount available) and incubating in an appropriate buffer (such as 0.3 M NaOAc, pH 6) at 90° C. for 30 seconds and then extracting with phenol/chloroform and chloroform. The resulting dsRNA can be precipitated with ethanol and dissolved in an appropriate buffer depending on the intended use of the dsRNA. These double-stranded siRNA structures can then be directly introduced to cells, either by passive uptake or a delivery system of choice.

In some embodiments, the eri-1 siRNA constructs can be generated by processing longer double-stranded RNAs, for example, in the presence of the enzyme dicer under conditions in which the dsRNA is processed to RNA molecules of about 21 to about 23 nucleotides.

In other embodiments, eri-1 RNA can be transcribed from PCR products, followed by gel purification. Standard procedures known in the art for in vitro transcription of RNA from PCR templates carrying, for example, T7 or SP6 promoter sequences can be used. The dsRNAs can be synthesized by using a PCR template and the AMBION (Austin, Tex.) T7 MEGASCRIPT kit, following the Manufacturer's recommendations and the RNA can then be precipitated with LiCl and resuspended in buffer. The specific dsRNAs produced can be tested for resistance to digestion by RNases A and T1. The dsRNAs can be produced with 3′ overhangs at both termini or one terminus of preferably 1-10 nucleotides, more preferably 1-3 nucleotides or with blunt ends at one or both termini. In one embodiment, thymidine nucleotide overhangs are useful for enhancing nuclease resistance of siRNAs.

Other standard methods for the preparation of siRNAs and other nucleobase oligomers are described, for example, in Ausubel et al., Current Protocols in Molecular Biology (Supplement 56), John Wiley & Sons, New York (2001); Sambrook and Russel, Molecular Cloning: A Laboratory Manual, 3rd ed., Cold Spring Harbor Press, Cold Spring Harbor (2001); and Dieffenbach and Dveksler, PCR Primer: A Laboratory Manual, Cold Spring Harbor Press (1995), all of which are incorporated herein by reference in their entirety.

eri-1 siRNA molecules can be purified using a number of techniques known to those of skill in the art. For example, gel electrophoresis can be used to purify eri-1 siRNAs. Alternatively, non-denaturing methods, such as non-denaturing column chromatography, can be used to purify the eri-1 siRNA. In addition, chromatography (e.g., size exclusion chromatography), glycerol gradient centrifugation, affinity purification with antibody can be used to purify siRNAs.

In preferred embodiments, at least one strand of the eri-1 siRNA molecules has a 3′ overhang from about 1 to about 6 nucleotides in length, though the overhang may be from 2 to 4 nucleotides in length. More preferably, the 3′ overhangs are 1-3 nucleotides in length. In other embodiments, one strand has a 3′ overhang and the other strand is blunt-ended or also has an overhang. The length of the overhangs may be the same or different for each strand. In order to further enhance the stability of the siRNA, the 3′ overhangs can be stabilized against degradation. In one embodiment, the eri-1 RNA is stabilized by including purine nucleotides, such as adenosine or guanosine nucleotides or by substituting pyrimidine nucleotides by modified analogs, e.g., substitution of uridine nucleotide 3′ overhangs by 2′-deoxythyinidine. In other embodiments, the absence of a 2′ hydroxyl can significantly enhance nuclease resistance of the overhang.

Also useful in the methods of the invention are eri-1 shRNAs. Such RNAs can be synthesized exogenously or can be formed by transcribing from a promoter in vivo. For expression of eri-1 shRNAs within cells, plasmid or viral vectors may contain, for example, a promoter, including, but not limited to the polymerase I, II, and III H1, U6, BL, SMK, 7SK, tRNA polIII, tRNA(met)-derived, and T7 promoters, a cloning site for the stem-looped RNA coding insert, and a 4-5-thymidine transcription termination signal. The Polymerase III promoters generally have well-defined initiation and stop sites and their transcripts lack poly(A) tails. Examples of making and using shRNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; and Yu et al., Proc Natl Acad Sci USA, 2002, 99:6047-52. Preferably, such shRNAs are engineered in cells or in an animal to ensure continuous and stable suppression of a target gene. It is known that siRNAs can be produced by processing a hairpin RNA in a cell.

siRNA Delivery

For some applications, a plasmid is used to deliver an eri-1 inhibitory nucleobase oligomer, such as a double stranded RNA, siRNA, or shRNA, as a transcriptional product. In such embodiments, the plasmid is designed to include a coding sequence for each of the sense and antisense strands of an eri-1 RNAi construct. The coding sequences can be the same sequence, e.g., flanked by inverted promoters, or can be two separate sequences each under the transcriptional control of separate promoters. After the coding sequence is transcribed, the complementary eri-1 RNA transcripts base pair to form a double-stranded RNA. PCT application WO01/77350 describes an exemplary vector for bi-directional transcription of a transgene to yield both sense and antisense RNA transcripts of the same transgene in a eukaryotic cell.

Methods for the production and therapeutic administration of siRNAs for in vivo therapies are described in U.S. Patent Application Publications: 20030180756, 20030157030, and 20030170891. Methods describing the successful in vivo use of siRNA are described by Song et al. (Nature Medicine 9: 347-351, 2003.

Administration to cells of eri-1 inhibitory nucleic acids, or vectors encoding such nucleic acids, can be carried out by any standard method. For example, an eri-1 inhibitory nucleic acid or a vector encoding an eri-1 inhibitory nucleic acid can be introduced in vivo by lipofection. Liposomes for encapsulation and transfection of nucleic acids in vitro may be used. For some applications, synthetic cationic lipids can be used to prepare liposomes for in vivo transfection (Felgner et. al., Proc. Natl. Acad. Sci. USA 84:7413-7417, 1987; See also, Mackey, et al., Proc. Natl. Acad. Sci. USA 85:8027-8031, 1988; Ulmer et al., Science 259:1745-1748, 1993). The use of cationic lipids may promote encapsulation of negatively charged nucleic acids, and also promote fusion with negatively charged cell membranes (Felgner and Ringold, Science 337:387-388, 1989). Particularly useful lipid compounds and compositions for transfer of nucleic acids are described in WO95/18863, WO96/17823, and in U.S. Pat. No. 5,459,127. Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce an eri-1 inhibitory nucleic acid or an expression vector encoding such a nucleic acid in vivo as a naked DNA. Methods for formulating and administering naked DNA to mammalian tissue are disclosed in U.S. Pat. Nos. 5,580,859 and 5,589,466.

Because inhibitory nucleic acids may be substrates for nuclease degradation, modified or substituted inhibitory nucleic acids are often preferred because of properties such as, for example, enhanced cellular uptake and increased stability in the presence of nucleases.

### Modified Nucleobase Oligomers

An eri-1 inhibitory nucleic acid or nucleobase oligomer may include modifications that increase nuclease resistance or that enhance the activity, cellular distribution or cellular uptake of the oligonucleotide. In various embodiments, an eri-1 oligomeric mimetic contains novel groups in place of the sugar, the backbone, or both. The base units are maintained to allow hybridization with an appropriate nucleic acid target compound.

Specific examples of some preferred eri-1 nucleic acids envisioned for this invention may contain phosphorothioates, phosphotriesters, methyl phosphonates, short chain alkyl or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Also preferred are oligonucleotides having morpholino backbone structures (Summerton, J. E. and Weller, D. D., U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen, M. Egholm, R. H. Berg, O Buchardt, Science 199, 254, 1497). Other preferred eri-1 oligonucleotides may contain alkyl and halogen-substituted sugar moieties comprising one of the following at the 2′ position: OH, SH, SCH₃, F, OCN, O(CH₂)ₙNH₂ or O(CH₂)ₙ CH₃, where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. eri-1 oligonucleotides may also have sugar mimetics such as cyclobutyls in place of the pentofuranosyl group.

In other preferred embodiments, an eri-1 oligomer may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines. In one embodiment, an eri-1 oligomer includes one or more G-clamp nucleotides. A G-clamp nucleotide is a modified cytosine analog having a modification that confers the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substitution within an oligomer can result in substantially enhanced helical thermal stability and mismatch discrimination when hybridized to complementary oligonucleotides. In another embodiment, eri-1 nucleic acid molecules of the invention include one or more LNA “locked nucleic acid” nucleotides such as a 2′,4′-C mythylene bicyclo nucleotide (see for example Wengel et al., International PCT Publication No. WO 00/66604 and WO 99/14226).

In other embodiments, an eri-1 oligomer contains one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the properties of an oligonucleotide include groups that improve oligomer uptake, enhance oligomer resistance to degradation, and/or strengthen sequence-specific hybridization with RNA, improve oligomer uptake, distribution, metabolism, or excretion. Conjugate moieties include, but are not limited to, lipid moieties such as a cholesterol moiety, cholic, a thioether, e.g., hexyl-5-tritylthiol, athiocholesterol, analiphatic chain, a phospholipid, e.g., di-hexadecyl-rac-glycerol or triethyl-ammonium1,2-di-O-hexadecyl-rac-glyc-ero-3-H-phosphonate, a polyamine or a polyethylene glycol chain, or adamantane acetic acid, a palmityl moiety, or an octadecylamine orhexylamino-carbonyl-oxycholesterol moiety. Methods for the preparation of such oligonucleotide conjugates are standard in the art, and include, but are not limited to exonuclease resistant terminally substituted oligonucleotides, which are described in U.S. Pat. No. 5,245,022; oligonucleotide-enzyme conjugates, which are described in U.S. Pat. No. 5,254,469; boronated phosphoramidate conjugates, which are described in U.S. Pat. No. 5,272,250; delectably tagged oligomers, which are described in U.S. Pat. No. 5,317,098; oligomer protein conjugates, which are described in U.S. Pat. No. 5,391,723; and steroid modified oligomers, which are described in U.S. Pat. No. 5,416,203. Other oligonucleotide conjugates are described in, for example, in U.S. Pat. Nos. 5,258,506; 5,262,536; 5,292,873; 5,371,241, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

eri-1 oligomers may also include nucleobase (often referred to in the art simply as “base”) modifications or substitutions. “Unmodified” or “natural” nucleotides include the purine bases adenine (A) and guanine (G), and the pyrimidine bases thymine (T), cytosine (C) and uracil (U). Modified nucleotides are known in the art, and are described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Modified nucleobases are particularly useful for increasing the binding affinity of the oligomeric compounds of the invention. These modified nucleobases include, but are not limited to, 5-substituted pyrimidines, 6-azapyrimidines and N-2, N-6 and 0-6 substituted purines, including 2-aminopropyladenine, 5-propynyluracil and S-propynylcytosine, 5-methylcytosine substitutions.

### Oligonucleotide Backbones

At least two types of oligonucleotides induce the cleavage of RNA by Rnase H: oligodeoxynucleotides with phosphodiester (PO) or phosphorothioate (PS) linkages. Although 2′-OMe-RNA sequences exhibit a high affinity for RNA targets, these sequences are not substrates for RNase H. A desirable oligonucleotide is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. Peptide Nucleic Acids (PNA) may also be employed.

### Locked Nucleic Acids

Locked nucleic acids (LNA) are nucleotide analogs that can be employed in the present invention. LNA contain a 2′O, 4′-C methylene bridge that restrict the flexibility of the ribofuranose ring of the nucleotide analog and locks it into the rigid bicyclic N-type conformation. LNA show improved resistance to certain exo- and endonucleases and activate RNAse H, making them suitable for use in methods described herein. LNA can be incorporated into almost any oligonucleotide. Moreover, LNA-containing oligonucleotides can be prepared using standard phosphoramidite synthesis protocols. Additional details regarding LNA can be found in PCT publication WO99/14226, hereby incorporated by reference.

### Arabinonucleic Acids

Arabinonucleic acids (ANA) can also be employed in the methods and reagents of the present invention. ANA are based on D-arabinose sugars instead of the natural D-2′-deoxyribose sugars. Underivatized ANA analogs have similar binding affinity for RNA as phosphorothioates. When the arabinose sugar is derivatized with fluorine (2′F-ANA), an enhancement in binding affinity results, and selective hydrolysis of bound RNA occurs efficiently in the resulting ANA/RNA and F-ANA/RNA duplexes. These analogs can be made stable in cellular media by a derivatization at their termini with simple L sugars.

Isolation of Additional eri-1 Genes

Based on the eri-1 nucleotide and amino acid sequences described herein, the isolation and identification of additional coding sequences of orthologous eri-1 genes is made possible using standard strategies and techniques that are well known in the art.

In one example, the ERI-1 polypeptides disclosed herein are used to search a database to identify orthologs, as described herein.

In another example, any one of the eri-1 nucleotide sequences described herein may be used in conventional methods of nucleic acid hybridization screening. Such hybridization techniques and screening procedures are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York. In one particular example, all or part of an eri-1 nucleic acid sequence may be used as a probe to screen a recombinant DNA library for genes having sequence identity to a eri-1 gene. Hybridizing sequences are detected by plaque or colony hybridization according to standard methods.

Alternatively, using all or a portion of an eri-1 nucleic acid sequence one may readily design gene- or nucleic acid sequence-specific oligonucleotide probes, including degenerate oligonucleotide probes (i.e., a mixture of all possible coding sequences for a given amino acid sequence). These oligonucleotides may be based upon the sequence of either DNA strand or any appropriate portion of the nucleic acid sequence. General methods for designing and preparing such probes are provided, for example, in Ausubel et al. (supra), and Berger and Kimmel, (Guide to Molecular Cloning Techniques, 1987, Academic Press, New York). These oligonucleotides are useful for eri-1 gene isolation, either through their use as probes capable of hybridizing to a eri-1 gene, or as complementary sequences or as primers for various amplification techniques, for example, polymerase chain reaction (PCR) cloning strategies. If desired, a combination of different, detectably-labelled oligonucleotide probes may be used for the screening of a recombinant DNA library. Such libraries are prepared according to methods well known in the art, for example, as described in Ausubel et al. (supra), or they may be obtained from commercial sources.

As discussed above, eri-1 sequence-specific oligonucleotides may also be used as primers in amplification cloning strategies, for example, using PCR. PCR methods are well known in the art and are described, for example, in PCR Technology, Erlich, ed., Stockton Press, London, 1989; PCR Protocols: A Guide to Methods and Applications, Innis et al., eds., Academic Press, Inc., New York, 1990; and Ausubel et al. (supra). Primers are optionally designed to allow cloning of the amplified product into a suitable vector, for example, by including appropriate restriction sites at the 5′ and 3′ ends of the amplified fragment (as described herein). If desired, nucleotide sequences may be isolated using the PCR “RACE” technique, or Rapid Amplification of cDNA Ends (see, e.g., Innis et al. (supra)). By this method, oligonucleotide primers based on a desired sequence are oriented in the 3′ and 5′ directions and are used to generate overlapping PCR fragments. These overlapping 3′- and 5′-end RACE products are combined to produce an intact full-length cDNA. This method is described in Innis et al. (supra); and Frohman et al., (Proc. Natl. Acad. Sci. USA 85:8998, 1988).

Partial sequences, e.g., sequence tags, are also useful as hybridization probes for identifying full-length sequences, as well as for screening databases for identifying previously unidentified related virulence genes.

In general, the invention includes any nucleic acid sequence that may be isolated as described herein or which is readily isolated by homology screening or PCR amplification using any of the nucleic acid sequences disclosed herein.

It will be appreciated by those skilled in the art that, as a result of the degeneracy of the genetic code, a multitude of polynucleotide sequences encoding eri-1 genes, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring eri-1 genes, and all such variations are to be considered as being specifically disclosed.

Although nucleotide sequences of eri-1 genes or their variants are preferably capable of hybridizing to the nucleotide sequence of a naturally occurring eri-1 genes under appropriately selected conditions of stringency, it may be advantageous to produce nucleotide sequences encoding eri-1 genes, or their derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding eri-1 genes and their derivatives without altering the encoded amino acid sequences include the production of RNA transcripts having more desirable properties, such as a greater half-life, than transcripts produced from the naturally occurring sequence.

The invention also encompasses production of DNA sequences that encode eri-1 genes, or fragments thereof generated entirely by synthetic chemistry. After production, the synthetic sequence may be inserted into any of the many available expression vectors and cell systems using reagents well known in the art. Moreover, synthetic chemistry may be used to introduce mutations into a sequence encoding any eri-1 gene, or any fragment thereof.

Also encompassed by the invention are polynucleotide sequences that are capable of hybridizing to any eri-1 polynucleotide sequences, and fragments thereof under various conditions of stringency. (See, e.g., Wahl, G. M. and S. L. Berger (1987) Methods Enzymol. 152:399; Kimmel, A. R. (1987) Methods Enzymol. 152:507) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The washing steps which follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

Methods for DNA sequencing are well known in the art and may be used to practice any of the embodiments of the invention. The resulting sequences are analyzed using a variety of algorithms which are well known in the art. (See, e.g., Ausubel, F. M. (1997) Short Protocols in Molecular Biology, John Wiley & Sons, New York N.Y., unit 7.7).

### Screening Assays

As discussed above, the identified ERI-1 polypeptides are siRNAses that inhibit RNAi. Based on this discovery, screening assays to identify compounds that decrease the nuclease activity of an ERI-1 polypeptide or that decrease the expression of an eri-1 nucleic acid sequence of the invention were developed. The method of screening may involve high-throughput techniques. In addition, these screening techniques may be carried out in cultured cells or in animals (such as nematodes).

Any number of methods are available for carrying out such screening assays. In one example, candidate compounds are added at varying concentrations to the culture medium of cultured cells or nematodes expressing one of the eri-1 nucleic acid sequences of the invention. eri-1 gene expression is then measured, for example, by standard Northern blot analysis (Ausubel et al., supra) or RT-PCR, using any appropriate fragment prepared from the nucleic acid molecule as a hybridization probe. The level of eri-1 gene expression in the presence of the candidate compound is compared to the level measured in a control culture medium lacking the candidate molecule. Such cultured cells include nematode cells (for example, C. elegans cells), mammalian, insect, or plant cells. A compound that inhibits eri-1 expression is considered useful in the invention; such a molecule may be used, for example, as a therapeutic to enhance RNAi.

In another example, the effect of candidate compounds is measured at the level of ERI-1 polypeptide production using the same general approach and standard immunological techniques, such as Western blotting or immunoprecipitation with an antibody specific for an ERI-1 polypeptide. For example, immunoassays may be used to detect or monitor the expression of at least one of the polypeptides of the invention in an organism. Polyclonal or monoclonal antibodies (produced as described above) that are capable of binding to such a polypeptide may be used in any standard immunoassay format (e.g., ELISA, Western blot, or RIA assay) to measure the level of the polypeptide. In another example, ERI-1 polypeptide expression is detected by fusing the ERI-1 polypeptide to a detectable reporter. A compound that reduces the expression of the polypeptide is considered particularly useful. Again, such a molecule may be used, for example, as a therapeutic to enhance RNAi.

In yet another working example, candidate compounds are screened for those that specifically bind to and antagonize an ERI-1 polypeptide. Particularly useful are those polypeptides that block binding of an ERI-1 active site to a nucleic acid substrate. The efficacy of such a candidate compound is dependent upon its ability to interact with ERI-1 or a functional equivalent thereof. Such an interaction can be readily assayed using any number of standard binding techniques and functional assays (e.g., those described in Ausubel et al., supra). For example, a candidate compound may be tested in vitro for interaction and binding with a polypeptide of the invention and its ability to enhance RNAi may be assayed by any standard assay (e.g., those described herein).

In one particular working example, a candidate compound that binds to an ERI-1 polypeptide, preferably the active site of an ERI-1 polypeptide, may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the ERI-1 polypeptide is identified on the basis of its ability to bind to the ERI-1 polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to enhance RNAi (e.g., as described herein). Compounds isolated by this approach may also be used, for example, as therapeutics to delay or ameliorate human diseases associated with the expression or overexpression of a gene. Compounds that are identified as binding to an ERI-1 polypeptide or an ERI-1 active site with an affinity constant less than or equal to 10 mM are considered particularly useful in the invention.

Potential antagonists include organic molecules, peptides, peptide mimetics, polypeptides, nucleic acids, and antibodies that bind to an eri-1 nucleic acid sequence or polypeptide of the invention and thereby decrease its nuclease activity. Potential antagonists also include small molecules that bind to and occupy the active site of the polypeptide thereby preventing binding to cellular binding molecules, such that normal biological activity is prevented.

Each of the eri-1 DNA sequences provided herein may also be used in the discovery and development of RNAi enhancing compounds. The encoded ERI-1 protein, upon expression, can be used as a target for the screening of RNAi enhancing drugs that inhibit ERI-1 protein activity. In one example, a drug screen is carried out in vitro, by contacting an eri-1 nucleic acid substrate (e.g., a double stranded nucleic acid molecule) with an ERI-1 polypeptide (e.g., human, plant, C. elegans, or pathogen) in the presence or absence of a candidate compound under conditions suitable for degradation of the substrate, as described by Dominski et al., (Mol Cell 12:295-305, 2003), and reduced degradation of the nucleic acid substrate is detected relative to the degradation present in a control assay carried out in the absence of the candidate compound. A compound that inhibits the degradation of the nucleic acid substrate is an eri-1 antagonist that is useful for enhancing RNAi.

In some embodiments, the nucleic acid substrate is a quenched fluorophore-nucleic acid covalent conjugate. Methods for preparing such conjugates are known to the skilled artisan, and are described, for example, by Trubetskoy et al. (Anal Biochem 300:22-6, 2002). In such screens, a nucleic acid is labeled with a fluorescent reagent using high fluorescent reagent/DNA input ratios that result in self-quenching of the fluorescent dye-nucleic acid covalent conjugate. Nuclease treatment of these conjugates results in de-quenching, i.e., an increase in fluorescence. A candidate compound that reduces the nuclease activity of an eri-1 polypeptide (e.g., a human, plant, pathogen, or C. elegans ERI-1) reduces de-quenching relative to the de-quenching observed in a corresponding nuclease assay not contacted with the candidate compound. Compounds that reduce the nuclease activity of an ERI-1 polypeptide are likely to be useful for enhancing RNAi.

Additionally, the DNA sequences encoding the amino terminal regions of the encoded protein or Shine-Delgarno or other translation facilitating sequences of the respective mRNA can be used to construct inhibitory nucleic acid sequences to control the expression of the coding sequence of interest. Such sequences may be isolated by standard techniques (Ausubel et al., supra).

The antagonists of the invention may be employed, for instance, to prevent, delay or ameliorate human or plant diseases associated with the expression or overexpression of a gene or to treat or prevent a pathogen infection.

Optionally, compounds identified in any of the above-described assays may be confirmed as useful in delaying or ameliorating human diseases associated in either standard tissue culture methods or animal models and, if successful, may be used as therapeutics for enhancing RNAi in a subject in need of gene silencing.

Small molecules of the invention preferably have a molecular weight below 2,000 daltons, more preferably between 300 and 1,000 daltons, and most preferably between 400 and 700 daltons. It is preferred that these small molecules are organic molecules.

### Test Compounds and Extracts

In general, compounds capable of enhancing RNAi are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in inhibiting nuclease activity should be employed whenever possible.

When a crude extract is found to have a RNAi enhancing activity, or an ERI-1 binding activity, further fractionation of the positive lead extract is necessary to isolate chemical constituents responsible for the observed effect. Thus, the goal of the extraction, fractionation, and purification process is the careful characterization and identification of a chemical entity within the crude extract having RNAi enhancing activity. Methods of fractionation and purification of such heterogenous extracts are known in the art. If desired, compounds shown to be useful agents for enhancing RNAi are chemically modified according to methods known in the art.

eri-1 RNAi in Plants

As described herein, eri-1 nucleic acid molecules and polypeptides are also expressed in plants. As in other eukaryotic cells, inhibitory eri-1 nucleic acids or nucleobase oligomers are useful in enhancing RNAi in a plant cell. RNAi provides a convenient mechanism for altering the phenotype of a plant by reducing or eliminating the expression of a particular endogenous target gene. It can also treat or prevent infection of a plant cell by a pathogen.

In one example, an inhibitory eri-1 nucleic acid molecule is administered to or expressed in a plant cell in conjunction with an inhibitory nucleic acid molecule that targets an endogenous gene of interest to enhance the silencing of that gene. In one example, an eri-1 siRNA enhances RNAi in a plant pathogen when used in conjunction with an siRNA that targets an essential plant pathogen gene. For some applications, an attenuated strain of a microorganism is engineered to express inhibitory nucleic acids that target a pathogen eri-1 gene and an essential pathogen gene. Exposure of the pathogen to the host plant results in ingestion of the RNAi microorganisms leading to the eri-1 enhanced silencing of the target pathogen gene. By enhancing the silencing of, for example, an essential pathogen gene, eri-1 prevents, reduces, or eliminates infection or infestation of the host plant by the pathogen.

For other applications, the inhibitory nucleic acid molecules are encapsulated in a synthetic matrix, such as a polymer, and applied to the surface of a host plant. Ingestion of host cells by a pathogen delivers the inhibitory molecules to the pathogen and results in the enhanced down-regulation of a target gene in the pathogen. Examples of plant pathogens include, but are not limited to viruses, bacteria, parasites, or insects in contact with the plant cell. Methods for using inhibitory nucleic acids in plants are known to the skilled artisan (see, for example, U.S. Pat. Nos. 6,452,067, 6,500,670, 6,395,962, 6,369,296, 6,002,071; or U.S. Patent Publication No. 20030150017).

### Construction of Plant Transgenes

Transgenic plants expressing an eri-1 transgene encoding an eri-1 inhibitory nucleic acid, including, but not limited to, dsRNA, siRNA, shRNA, or antisense RNA, are useful for enhancing RNAi in a plant. A transgenic plant, or population of such plants, expressing at least one eri-1 transgene encoding an eri-1 inhibitory nucleic acid would be expected to show an enhanced response to RNAi. For some applications, an eri-1 inhibitory nucleic acid molecule is co-expressed with a transgene encoding an inhibitory nucleic acid molecule that targets a gene of interest. In plants, as in mammals, eri-1 RNAi is useful in enhancing the silencing of virtually any endogenous or pathogen gene of interest.

In one preferred embodiment, an eri-1 inhibitory nucleic acid (e.g., double-stranded RNA, siRNA, or antisense RNA) is expressed by a stably-transfected plant cell line, a transiently-transfected plant cell line, or by a transgenic plant. A number of vectors suitable for stable or extrachromosomal transfection of plant cells or for the establishment of transgenic plants are available to the public; such vectors are described in Pouwels et al. (supra), Weissbach and Weissbach (supra), and Gelvin et al. (supra). Methods for constructing such cell lines are described in, e.g., Weissbach and Weissbach (supra), and Gelvin et al. (supra).

Vectors useful in the methods of the invention are described, for example, in U.S. Pat. No. 5,922,602, WO 99/36516, Virology 267:29-35, 2000, and U.S. Patent Publication No. 20020165370.

Plant expression constructs having an eri-1 gene that encodes an eri-1 inhibitory nucleic acid may be employed with a wide variety of plant life, particularly plant life involved in the production of storage reserves (for example, those involving carbon and nitrogen metabolism). Such genetically-engineered plants are useful for a variety of industrial and agricultural applications. Importantly, this invention is applicable to dicotyledons and monocotyledons, and will be readily applicable to any new or improved transformation or regeneration method.

The expression constructs include at least one promoter operably linked to at least one eri-1 gene (e.g., encoding an eri-1 inhibitory nucleic acid). Examples of plant expression constructs are found in Fraley et al., U.S. Pat. No. 5,352,605. In most tissues of transgenic plants, the CaMV 35S promoter is a strong promoter (see, e.g., Odell et al., Nature 313:810, 1985). Other useful plant promoters include, without limitation, the nopaline synthase (NOS) promoter (An et al., Plant Physiol. 88:547, 1988 and Rodgers and Fraley, U.S. Pat. No. 5,034,322), the octopine synthase promoter (Fromm et al., Plant Cell 1:977, 1989), figwort mosiac virus (FMV) promoter (Rodgers, U.S. Pat. No. 5,378,619), and the rice actin promoter (Wu and McElroy, WO91/09948).

Exemplary monocot promoters include, without limitation, commelina yellow mottle virus promoter, sugar cane badna virus promoter, rice tungro bacilliform virus promoter, maize streak virus element, and wheat dwarf virus promoter.

For certain applications, it may be desirable to produce eri-1 inhibitory nucleic acid in an appropriate tissue, at an appropriate level, or at an appropriate developmental time. For this purpose, there are an assortment of gene promoters, each with its own distinct characteristics embodied in its regulatory sequences, shown to be regulated in response to inducible signals such as the environment, hormones, and/or developmental cues. These include, without limitation, gene promoters that are responsible for heat-regulated gene expression (see, e.g., Callis et al., Plant Physiol. 88:965, 1988; Takahashi and Komeda, Mol. Gen. Genet. 219:365, 1989; and Takahashi et al. Plant J. 2:751, 1992), light-regulated gene expression (e.g., the pea rbcS-3A described by Kuhlemeier et al., Plant Cell 1:471, 1989; the maize rbcS promoter described by Schäffner and Sheen, Plant Cell 3:997, 1991; the chlorophyll a/b-binding protein gene found in pea described by Simpson et al., EMBO J. 4:2723, 1985; the Arabssu promoter; or the rice rbs promoter), hormone-regulated gene expression (for example, the abscisic acid (ABA) responsive sequences from the Em gene of wheat described by Marcotte et al., Plant Cell 1:969, 1989; the ABA-inducible HVA1 and HVA22, and rd29A promoters described for barley and Arabidopsis by Straub et al., Plant Cell 6:617, 1994 and Shen et al., Plant Cell 7:295, 1995; and wound-induced gene expression (for example, of wunI described by Siebertz et al., Plant Cell 1:961, 1989), organ-specific gene expression (for example, of the tuber-specific storage protein gene described by Roshal et al., EMBO J. 6:1155, 1987; the 23-kDa zein gene from maize described by Schernthaner et al., EMBO J. 7:1249, 1988; or the French bean β-phaseolin gene described by Bustos et al., Plant Cell 1:839, 1989), or pathogen-inducible promoters (for example, PR-1, prp-1, or -1,3 glucanase promoters, the fungal-inducible wirla promoter of wheat, and the nematode-inducible promoters, TobRB7-5A and Hmg-1, of tobacco and parsley, respectively).

Plant expression vectors may also optionally include RNA processing signals, e.g., introns, which have been shown to be important for efficient RNA synthesis and accumulation (Callis et al., Genes and Dev. 1: 1183, 1987). The location of the RNA splice sequences can dramatically influence the level of transgene expression in plants. In view of this fact, an intron may be positioned upstream or downstream of an eri-1 inhibitory nucleic acid-encoding sequence in the transgene to modulate levels of gene expression.

In addition to the aforementioned 5′ regulatory control sequences, the expression vectors may also include regulatory control regions which are generally present in the 3′ regions of plant genes (Thornburg et al., Proc. Natl. Acad. Sci. U.S.A. 84:744, 1987; An et al., Plant Cell 1:115, 1989). For example, the 3′ terminator region may be included in the expression vector to increase stability of the mRNA. One such terminator region may be derived from the PI-II terminator region of potato. In addition, other commonly used terminators are derived from the octopine or nopaline synthase signals.

The plant expression vector also typically contains a dominant selectable marker gene used to identify those cells that have become transformed. Useful selectable genes for plant systems include genes encoding antibiotic resistance genes, for example, those encoding resistance to hygromycin, kanamycin, bleomycin, G418, streptomycin, or spectinomycin. Genes required for photosynthesis may also be used as selectable markers in photosynthetic-deficient strains. Finally, genes encoding herbicide resistance may be used as selectable markers; useful herbicide resistance genes include the bar gene encoding the enzyme phosphinothricin acetyltransferase and conferring resistance to the broad spectrum herbicide Basta® (Frankfurt, Germany).

Efficient use of selectable markers is facilitated by a determination of the susceptibility of a plant cell to a particular selectable agent and a determination of the concentration of this agent which effectively kills most, if not all, of the transformed cells. Some useful concentrations of antibiotics for tobacco transformation include, e.g., 75-100 μg/mL (kanamycin), 20-50 μg/mL (hygromycin), or 5-10 μg/mL (bleomycin). A useful strategy for selection of transformants for herbicide resistance is described, e.g., by Vasil et al., supra.

In addition, if desired, the plant expression construct may contain a modified or fully-synthetic structural eri-1 inhibitory nucleic acid coding sequence that has been changed to enhance the performance of the gene in plants. Methods for constructing such a modified or synthetic gene are described in Fischoff and Perlak, U.S. Pat. No. 5,500,365.

It should be readily apparent to one skilled in the art of molecular biology, especially in the field of plant molecular biology, that the level of gene expression is dependent, not only on the combination of promoters, RNA processing signals, and terminator elements, but also on how these elements are used to increase the levels of selectable marker gene expression.

### Plant Transformation

Upon construction of the plant expression vector, several standard methods are available for introduction of the vector into a plant host, thereby generating a transgenic plant. These methods include (1) Agrobacterium-mediated transformation (A. tumefaciens or A. rhizogenes) (see, e.g., Lichtenstein and Fuller In: Genetic Engineering, vol 6, P W J Rigby, ed, London, Academic Press, 1987; and Lichtenstein, C. P., and Draper, J,. In: DNA Cloning, Vol II, D. M. Glover, ed, Oxford, IRI Press, 1985); (2) the particle delivery system (see, e.g., Gordon-Kamm et al., Plant Cell 2:603, 1990); or BioRad Technical Bulletin 1687, supra), (3) microinjection protocols, (4) polyethylene glycol (PEG) procedures (see, e.g., Draper et al., Plant Cell Physiol. 23:451, 1982; or e.g., Zhang and Wu, Theor. Appl. Genet. 76:835, 1988), (5) liposome-mediated DNA uptake (see, e.g., Freeman et al., Plant Cell Physiol. 25:1353, 1984), (6) electroporation protocols (see, e.g., Fromm et al., Nature 319:791, 1986; Sheen Plant Cell 2:1027, 1990; or Jang and Sheen Plant Cell 6:1665, 1994), and (7) the vortexing method (see, e.g., Kindle supra). The method of transformation is not critical to the invention. Any method that provides for efficient transformation may be employed. As newer methods are available to transform crops or other host cells, they may be directly applied. Suitable plants for use in the practice of the invention include, but are not limited to, sugar cane, wheat, rice, maize, sugar beet, potato, barley, manioc, sweet potato, soybean, sorghum, cassava, banana, grape, oats, tomato, millet, coconut, orange, rye, cabbage, apple, watermelon, canola, cotton, carrot, garlic, onion, pepper, strawberry, yam, peanut, onion, bean, pea, mango, citrus plants, walnuts, and sunflower.

The following is an example outlining one particular technique, an Agrobacterium-mediated plant transformation. By this technique, the general process for manipulating genes to be transferred into the genome of plant cells is carried out in two phases. First, cloning and DNA modification steps are carried out in E. coli, and the plasmid containing the gene construct of interest is transferred by conjugation or electroporation into Agrobacterium. Second, the resulting Agrobacterium strain is used to transform plant cells. Thus, for the generalized plant expression vector, the plasmid contains an origin of replication that allows it to replicate in Agrobacterium and a high copy number origin of replication functional in E. coli. This permits facile production and testing of transgenes in E. coli prior to transfer to Agrobacterium for subsequent introduction into plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer functions of Agrobacterium, delimit the DNA region that will be transferred to the plant.

In another example, plant cells may be transformed by shooting into the cell tungsten microprojectiles on which cloned DNA is precipitated. In the Biolistic Apparatus (Bio-Rad) used for the shooting, a gunpowder charge (22 caliber Power Piston Tool Charge) or an air-driven blast drives a plastic macroprojectile through a gun barrel. An aliquot of a suspension of tungsten particles on which DNA has been precipitated is placed on the front of the plastic macroprojectile. The latter is fired at an acrylic stopping plate that has a hole through it that is too small for the macroprojectile to pass through. As a result, the plastic macroprojectile smashes against the stopping plate, and the tungsten microprojectiles continue toward their target through the hole in the plate. For the instant invention the target can be any plant cell, tissue, seed, or embryo. The DNA introduced into the cell on the microprojectiles becomes integrated into either the nucleus or the chloroplast.

In general, the transfer and expression of transgenes in plant cells is now routine for one skilled in the art, and have become major tools to carry out gene expression studies in plants and to produce improved plant varieties of agricultural or commercial interest.

### Transgenic Plant Regeneration

Plant cells transformed with a plant expression vector can be regenerated, for example, from single cells, callus tissue, or leaf discs according to standard plant tissue culture techniques. It is well known in the art that various cells, tissues, and organs from almost any plant can be successfully cultured to regenerate an entire plant.

In one particular example, a cloned eri-1 inhibitory nucleic acid expression construct under the control of the 35S CaMV promoter and the nopaline synthase terminator and carrying a selectable marker (for example, kanamycin resistance) is transformed into Agrobacterium. Transformation of leaf discs, with vector-containing Agrobacterium is carried out as described by Horsch et al. (Science 227:1229, 1985). Putative transformants are selected after a few weeks (for example, 3 to 5 weeks) on plant tissue culture media containing kanamycin (e.g. 100 μg/mL). Kanamycin-resistant shoots are then placed on plant tissue culture media without hormones for root initiation. Kanamycin-resistant plants are then selected for greenhouse growth. If desired, seeds from self-fertilized transgenic plants can then be sowed in a soil-less medium and grown in a greenhouse. Kanamycin-resistant progeny are selected by sowing surfaced sterilized seeds on hormone-free kanamycin-containing media. Analysis for the integration of the transgene is accomplished by standard techniques (see, for example, Ausubel et al. supra; Gelvin et al. supra).

Transgenic plants expressing the selectable marker are then screened for transmission of the transgene DNA by standard immunoblot and DNA detection techniques. Each positive transgenic plant and its transgenic progeny are unique in comparison to other transgenic plants established with the same transgene. Integration of the transgene DNA into the plant genomic DNA is in most cases random, and the site of integration can profoundly affect the levels and the tissue and developmental patterns of transgene expression. Consequently, a number of transgenic lines are usually screened for each transgene to identify and select plants with the most appropriate expression profiles.

Transgenic lines are evaluated for levels of transgene expression. Expression at the RNA level is determined initially to identify and quantitate expression-positive plants. Standard techniques for RNA analysis are employed for transgenic plants expressing eri-1 inhibitory nucleobase oligomers. Such techniques include PCR amplification assays using oligonucleotide primers designed to amplify only transgene RNA templates and solution hybridization assays using transgene-specific probes (see, e.g., Ausubel et al., supra). Those RNA-positive plants that encode an eri-1 inhibitory nucleic acid are then analyzed for protein expression by Western immunoblot analysis using specific antibodies (see, e.g., Ausubel et al., supra) to detect a decrease in the level of expression of a gene of interest. In addition, immunocytochemistry according to standard protocols can be done using specific antibodies to detect a decrease in the level of expression of a target gene within transgenic tissue.

Ectopic expression of one or more eri-1 inhibitory nucleobase oligomer is useful for the production of transgenic plants that exhibit enhanced RNAi.

Transgenic Plants Expressing an eri-1 Inhibitory Nucleic Acid

As discussed above, plasmid constructs designed for the expression of eri-1 inhibitory nucleobase oligomers (e.g., double-stranded RNA, siRNA, or antisense RNA) are useful for enhancing RNAi in a transgenic plant.

eri-1 inhibitory nucleic acids may be engineered for expression in a plant. An eri-1 dsRNA may be expressed in its entirety, or a portion of the eri-1 dsRNA may be expressed. The portion (e.g., 10%, 20%, 30%, 40%, 50%, 60%, 70%, 80%, 90%, or even 95%) of the full length nucleic acid may be selected to maximize specificity. To enhance RNAi in a transgenic plant, it is important to express a dsRNA eri-1 at an effective level. Evaluation of the level of enhanced RNAi conferred to a plant by ectopic expression of a dsRNA eri-1 is determined according to conventional methods and assays.

In one embodiment, constitutive ectopic expression of an inhibitory eri-1 nucleic acid is generated by transforming a plant with a plant expression vector containing a nucleic acid sequence encoding an inhibitory eri-1 nucleic acid (e.g., double stranded RNA, antisense RNA, siRNA, or shRNA). This expression vector is then used to transform a plant according to standard methods known to the skilled artisan and described in Fischhoff et al. (U.S. Pat. No. 5,500,365).

The frequency with which post-transcriptional gene silencing is obtained in a population of plants, each of which is the result of an independent transformation event, can range widely, from less than 1% to 30% or more. A screening step is therefore useful in the production of plants which exhibit post-transcriptional gene silencing. Several screening methods have been used to select from a transgenic plant population those plants in which expression of a targeted gene is suppressed. These screening methods include, but are not limited to, visual screening of a suitable trait (e.g., flower color); quantitation of the final product of a biosynthetic pathway that includes the protein product of the targeted gene as a pathway enzyme; quantitation of the protein product of the target gene; quantitation of the mRNA product of the target gene, using Northern analysis, RNase protection assay, RT-PCR, or other suitable technique; quantitation of the transgene mRNA in vegetative tissue using Northern analysis or other suitable technique.

The invention further provides for increased production efficiency, as well as for improvements in quality and yield of crop plants and ornamentals. Thus, the invention contributes to the production of high quality and high yield agricultural products, for example, fruits, ornamentals, vegetables, cereals and field crops having reduced spots, blemishes, and blotches that are caused by insects or nematodes; agricultural products with increased shelf-life and reduced handling costs; and high quality and yield crops for agricultural (for example, cereal and field crops), industrial (for example, oilseeds), and commercial (for example, fiber crops) purposes. Furthermore, because the invention reduces the necessity for chemical protection against plant pathogens, the invention benefits the environment where the crops are grown. Genetically-improved seeds and other plant products that are produced using plants expressing the nucleic acids described herein also render farming possible in areas previously unsuitable for agricultural production.

### Use of Transgenic and Knockout Animals in Diagnosis or Drug Screening

The present invention also includes transgenic animals expressing eri-1 inhibitory nucleic acids and eri-1 knock-out animals that exhibit enhanced RNAi. Such animals are useful to determine genetic and physiological features of RNAi or to study the biological activity of a polypeptide by inhibiting the expression of the polypeptide using RNAi.

Transgenic animals include animals expressing a dsRNA that targets an endogenous eri-1 nucleic acid sequence. Because such transgenic animals and eri-1 knock-out animals likely express decreased levels of eri-1, relative to a wild-type control animal, they are likely to exhibit enhanced RNAi, and are useful for the analysis of genes that are refractory to RNAi.

In one example, RNAi is used to target eri-1 and an endogenous gene of interest where RNAi is used to generate an animal model for disease. Typically, such a disease is a monogenic disease, where deletion of a single gene or mutation is sufficient to induce a disease phenotype, such as cystic fibrosis, Duchenne muscular dystrophy, hemophilia, adenosine deaminase deficiency, or familial hypercholesteremia.

Knockout animals that are either homozygous or heterozygous, for a deletion in an eri-1 nucleic acid molecule are also expected to exhibit enhanced RNAi. Knockout animals include animals where the normal eri-1 gene has been inactivated, deleted, or replaced with a mutant allele of an endogenous eri-1 gene.

In general, methods of detecting a transgenic or knockout animal having enhanced RNAi involve comparing the level of expression of an eri-1 gene, either at the RNA level or at the protein level, in tissue from a transgenic or knock-out animal and in tissue from a matching non-transgenic or non-knock-out animal. Standard techniques for detecting RNA expression, e.g., by Northern blotting, or protein expression, e.g., by Western blotting, are well known in the art.

For some applications, an animal displaying enhanced RNAi (e.g., an animal expressing an eri-1 inhibitory nucleic acid or having a deletion or inactivation in eri-1) is contacted with an inhibitory nucleobase oligomer that targets a gene of interest, and then the expression of at least one or more other genes is detected. Differences between an animal contacted with an inhibitory nucleobase oligomer and a control animal, such as the presence, absence, or level of expression of at least one or more genes indicates that the expression of such a gene is regulated by the gene targeted for RNAi.

Patterns of accumulation or reduction of a variety of nucleic acid molecules can be surveyed using, for example, a microarray. Screens directed at analyzing expression of specific genes or groups of molecules can be continued during the life of the eri-1 inhibitory nucleic acid expressing transgenic animal or eri-1 knockout animal. Protein expression can be monitored by immunohistochemistry as well as by protein microarray and RNA blotting techniques.

An eri-1 knockout organism may be a conditional, i.e., somatic, knockout. For example, FRT sequences may be introduced into the organism so that they flank the gene of interest. Transient or continuous expression of the FLP protein may then be used to induce site-directed recombination, resulting in the excision of an eri-1 gene. The use of the FLP/FRT system is well established in the art and is described in, for example, U.S. Pat. No. 5,527,695, and in Lyznik et al. (Nucleic Acid Research 24:3784-3789, 1996).

Conditional, i.e., somatic knockout organisms may also be produced using the Cre-lox recombination system. Cre is an enzyme that excises DNA between two recognition sites termed loxP. The cre transgene may be under the control of an inducible, developmentally regulated, tissue specific, or cell-type specific promoter. In the presence of Cre, the gene, for example a nucleic acid sequence described herein, flanked by loxP sites is excised, generating a knockout. This system is described, for example, in Kilby et al. (Trends in Genetics 9:413-421, 1993).

Particularly desirable is a mouse model where a dsRNA targeting a gene of interest, such as eri-1 is expressed in specific cells of the transgenic mouse such that those cells exhibit enhanced RNAi. In addition, cell lines from these mice may be established by methods standard in the art.

Construction of transgenes can be accomplished using any suitable genetic engineering technique, such as those described in Ausubel et al. (Current Protocols in Molecular Biology, John Wiley & Sons, New York, 2000). Many techniques of transgene construction and of expression constructs for transfection or transformation in general are known and may be used for the disclosed constructs.

One skilled in the art will appreciate that a promoter is chosen that directs expression of an eri-1 inhibitory nucleic acid in a tissue that requires gene silencing. For example, as noted above, any promoter that regulates expression of a nucleic acid sequence described herein can be used in the expression constructs of the present invention. One skilled in the art would be aware that the modular nature of transcriptional regulatory elements and the absence of position-dependence of the function of some regulatory elements, such as enhancers, make modifications such as, for example, rearrangements, deletions of some elements or extraneous sequences, and insertion of heterologous elements possible. Numerous techniques are available for dissecting the regulatory elements of genes to determine their location and function. Such information can be used to direct modification of the elements, if desired. It is desirable, however, that an intact region of the transcriptional regulatory elements of a gene is used. Once a suitable transgene construct has been made, any suitable technique for introducing this construct into embryonic cells can be used.

Animals suitable for transgenic experiments can be obtained from standard commercial sources such as Taconic (Germantown, N.Y.). Many strains are suitable, but Swiss Webster (Taconic) female mice are desirable for embryo retrieval and transfer. B6D2F (Taconic) males can be used for mating and vasectomized Swiss Webster studs can be used to stimulate pseudopregnancy. Vasectomized mice and rats are publicly available from the above-mentioned suppliers. However, one skilled in the art would also know how to make a transgenic mouse or rat. An example of a protocol that can be used to produce a transgenic animal is provided below.

### Production of Transgenic Mice and Rats

The following is but one desirable means of producing transgenic mice. This general protocol may be modified by those skilled in the art.

Female mice six weeks of age are induced to superovulate with a 5 IU injection (0.1 cc, IP) of pregnant mare serum gonadotropin (PMSG; Sigma) followed 48 hours later by a 5 IU injection (0.1 cc, IP) of human chorionic gonadotropin (hCG, Sigma). Females are placed together with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA, Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with humidified atmosphere at 5% CO₂, 95% air until the time of injection. Embryos can be implanted at the two-cell stage.

Randomly cycling adult female mice are paired with vasectomized males. Swiss Webster or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmaker's forceps. Embryos to be transferred are placed in DPBS (Dulbecco's phosphate buffered saline) and in the tip of a transfer pipet (about 10 to 12 embryos). The pipet tip is inserted into the infundibulum and the embryos are transferred. After the transferring the embryos, the incision is closed by two sutures.

A desirable procedure for generating transgenic rats is similar to that described above for mice (Hammer et al., Cell 63:1099-112, 1990). For example, thirty-day old female rats are given a subcutaneous injection of 20 IU of PMSG (0.1 cc) and 48 hours later each female placed with a proven, fertile male. At the same time, 40-80 day old females are placed in cages with vasectomized males. These will provide the foster mothers for embryo transfer. The next morning females are checked for vaginal plugs. Females who have mated with vasectomized males are held aside until the time of transfer. Donor females that have mated are sacrificed (CO₂ asphyxiation) and their oviducts removed, placed in DPBA (Dulbecco's phosphate buffered saline) with 0.5% BSA and the embryos collected. Cumulus cells surrounding the embryos are removed with hyaluronidase (1 mg/ml). The embryos are then washed and placed in EBSs (Earle's balanced salt solution) containing 0.5% BSA in a 37.5° C. incubator until the time of microinjection.

Once the embryos are injected, the live embryos are moved to DPBS for transfer into foster mothers. The foster mothers are anesthetized with ketamine (40 mg/kg, IP) and xulazine (5 mg/kg, IP). A dorsal midline incision is made through the skin and the ovary and oviduct are exposed by an incision through the muscle layer directly over the ovary. The ovarian bursa is torn, the embryos are picked up into the transfer pipet, and the tip of the transfer pipet is inserted into the infundibulum. Approximately 10 to 12 embryos are transferred into each rat oviduct through the infundibulum. The incision is then closed with sutures, and the foster mothers are housed singly.

### Generation of Knockout Mice

The following is but one example for the generation of a knockout mouse and the protocol may be readily adapted or modified by those skilled in the art.

Embryonic stem cells (ES), for example, 10⁷ AB1 cells, may be electroporated with 25 μg targeting construct in 0.9 ml PBS using a Bio-Rad Gene Pulser (500 μF, 230 V). The cells may then be plated on one or two 10-cm plates containing a monolayer of irradiated STO feeder cells. Twenty-four hours later, they may be subjected to G418 selection (350 μg/ml, Gibco) for 9 days. Resistant clones may then be analyzed by Southern blotting after Hind III digestion, using a probe specific to the targeting construct. Positive clones are expanded and injected into C57BL/6 blastocysts. Male chimeras may be back-crossed to C57BL/6 females. Heterozygotes may be identified by Southern blotting and intercrossed to generate homozygotes.

The targeting construct may result in the disruption of the gene of interest, e.g., by insertion of a heterologous sequence containing stop codons, or the construct may be used to replace the wild-type gene with a mutant form of the same gene, e.g., a “knock-in.” Furthermore, the targeting construct may contain a sequence that allows for conditional expression of the gene of interest. For example, a sequence may be inserted into the gene of interest that results in the protein not being expressed in the presence of tetracycline. Such conditional expression of a gene is described in, for example, Yamamoto et al. (Cell 101:57-66, 2000).

### Microarrays

The global analysis of gene expression using gene chips can provide insights into gene expression perturbations in cells, tissues, or organisms administered dsRNAs. Such analysis can now be carried out in cells, tissues, or organisms that fail to express functional eri-1. Such methods allow for the analysis of genes that are refractory to conventionally used methods of RNAi. In addition, by enhancing the efficiency of RNAi, such methods increase the sensitivity of gene expression analysis for virtually any gene targeted for RNAi.

Microarrays may be prepared, used, and analyzed using methods known in the art. (See, e.g., Brennan et al., U.S. Pat. No. 5,474,796; Schena et al., Proc. Natl. Acad. Sci. 93:10614, 1996; Baldeschweiler et al., PCT application WO95/251116, 1995; Shalon, D. et al., PCT application WO95/35505, 1995; Heller et al., Proc. Natl. Acad. Sci. 94:2150, 1997; and Heller et al., U.S. Pat. No. 5,605,662.)

In general, hybridizable array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28:e3.i-e3.vii, 2000), MacBeath et al., (Science 289:1760-1763, 2000), Zhu et al. (Nature Genet. 26:283-289), and in U.S. Pat. No. 6,436,665, hereby incorporated by reference.

Nucleic Acid Microarrays

To produce a nucleic acid microarray oligonucleotides may be synthesized or bound to the surface of a substrate using a chemical coupling procedure and an ink jet application apparatus, as described in PCT application WO95/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample, such as a cultured cell, a tissue specimen, or other source, may be used to produce a hybridization probe as described herein. The mRNA is isolated according to standard methods, and cDNA is produced and used as a template to make complementary RNA suitable for hybridization using standard methods. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

A detection system may be used to measure the absence, presence, and amount of hybridization for all of the distinct sequences simultaneously (e.g., Heller et al., Proc. Natl. Acad. Sci. 94:2150-2155, 1997). Preferably, a scanner is used to determine the levels and patterns of fluorescence.

Protein Microarrays

ERI-1 proteins, such as those described herein, may be analyzed using protein microarrays. Such arrays are useful in high-throughput low-cost screens to identify peptide or candidate compounds that bind an ERI-1 polypeptide of the invention, or fragment thereof. Typically, protein microarrays feature a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., polypeptides of interest or antibodies against such polypeptides) are spotted on a substrate using any convenient method known to the skilled artisan (e.g., by hand or by inkjet printer). Preferably, such methods retain the biological activity or function of the protein bound to the substrate (Ge et al., supra; Zhu et al., supra).

The protein microarray is hybridized with a detectable probe. Such probes can be polypeptide, nucleic acid, or small molecules. For some applications, polypeptide and nucleic acid probes are derived from a biological sample taken from a patient, such as a bodily fluid (such as blood, urine, saliva, or phlegm); a homogenized tissue sample (e.g. a tissue sample obtained by biopsy); or cultured cells (e.g., lymphocytes). Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

### OTHER EMBODIMENTS

From the foregoing description, it will be apparent that variations and modifications may be made to the invention described herein to adapt it to various usages and conditions. Such embodiments are also within the scope of the following claims.

All publications mentioned in this specification are herein incorporated by reference to the same extent as if each independent publication was specifically and individually indicated to be incorporated by reference.

What is claimed is:

1. A method for identifying a mutation in a nucleic acid molecule encoding a polypeptide that inhibits RNA interference (RNAi), said method comprising:
(a) providing a mutagenized nematode comprising a gene that is expressed in a cell that is refractory to RNAi;
(b) contacting said nematode with an inhibitory nucleobase oligomer that targets said gene; and
(c) detecting a decrease in the expression of said gene in said mutagenized nematode relative to a control nematode, wherein a mutation in a nucleic acid molecule encoding a polypeptide that inhibits RNAi is identified by said decrease in the expression of said targeted gene.

2. The method of claim 1, wherein said decrease is detected by monitoring the expression of a reporter gene.

3. The method of claim 1, wherein said cell is a neuron.

4. The method of claim 1, wherein said inhibitory nucleobase oligomer is a dsRNA, siRNA, or dsRNA mimetic.

5. The method of claim 1, wherein said mutation identifies said nucleic acid molecule.

6. A method for identifying a mutation in a nucleic acid molecule encoding a polypeptide that inhibits RNAi, said method comprising:
(a) providing a mutagenized cell expressing a gene that is refractory to RNAi;
(b) contacting said cell with an inhibitory nucleobase oligomer that targets said refractory gene; and
(c) detecting a decrease in the expression of said gene, wherein a mutation in a nucleic acid molecule encoding a polypeptide that inhibits RNAi is identified by detecting said decrease.

7. The method of claim 6, wherein said cell is a nematode cell.

8. The method of claim 6, wherein said cell is a mammalian cell.

9. The method of claim 6, wherein said decrease is detected by monitoring the expression of a reporter gene.

10. The method of claim 6, wherein said mutation identifies said nucleic acid molecule.

11. A method for identifying a candidate compound that enhances RNAi, said method comprising:
(a) providing a cell expressing an eri-1 nucleic acid molecule;
(b) contacting said cell with a candidate compound; and
(c) comparing the expression of said eri-1 nucleic acid molecule in said cell contacted with said candidate compound with the expression of said eri-1 nucleic acid molecule in a control cell, wherein a decrease in said expression identifies said candidate compound as a candidate compound that enhances RNAi.

12. The method of claim 11, wherein said screening method identifies a compound that decreases transcription of said nucleic acid molecule.

13. The method of claim 11, wherein said screening method identifies a compound that decreases translation of an mRNA transcribed from said nucleic acid molecule.

14. The method of claim 11, wherein the compound is a member of a chemical library.

15. The method of claim 11, wherein said cell is in a nematode.

16. A method for identifying a candidate compound that enhances RNAi, said method comprising:
(a) providing a cell expressing an ERI-1 polypeptide;
(b) contacting said cell with a candidate compound; and
(c) comparing the biological activity of said ERI-1 polypeptide in said cell contacted with said candidate compound to a control cell, wherein a decrease in said biological activity of said ERI-1 polypeptide identifies said candidate compound as a candidate compound that enhances RNAi.

17. The method of claim 16, wherein said cell is a nematode cell.

18. The method of claim 17, wherein said cell is in a nematode.

19. The method of claim 16, wherein said cell is a mammalian cell.

20. The method of claim 16, wherein said cell is a plant cell.

21. The method of claim 16, wherein said ERI-1 polypeptide is an endogenous polypeptide.

22. The method of claim 16, wherein said biological activity is monitored with an enzymatic assay.

23. The method of claim 16, wherein said biological activity is monitored with an immunological assay.

24. The method of claim 16, wherein said biological activity is monitored by detecting degradation of an ERI-1 nucleic acid substrate.

25. The method of claim 23, wherein said nucleic acid substrate is an siRNA.

26. A method for identifying a candidate compound that enhances RNAi, said method comprising:
(a) providing an ERI-1 polypeptide;
(b) contacting said polypeptide with a candidate compound; and
(c) detecting binding of said ERI-1 polypeptide and said candidate compound, wherein a compound that binds to said ERI-1 polypeptide is a candidate compound that enhances RNAi.

27. The method of claim 23, wherein said candidate compound binds to and blocks an ERI-1 active site.

28. A method for identifying a candidate compound that enhances RNAi, said method comprising:
(a) providing an ERI-1 polypeptide and a nucleic acid substrate;
(b) contacting said ERI-1 polypeptide and said nucleic acid substrate with a candidate compound under conditions suitable for substrate degradation; and
(c) detecting a decrease in substrate degradation in the presence of said candidate compound relative to substrate degradation in the absence of said candidate compound, wherein a decrease in said substrate degradation identifies said candidate compound as a candidate compound that enhances RNAi.

29. The method of claim 28, wherein said nucleic acid substrate is an siRNA.

30. The method of claim 28, wherein said nucleic acid substrate is coupled to a fluorophore.

31. A method for identifying a candidate compound that enhances RNAi, said method comprising:
(a) providing a cell expressing an ERI-1 polypeptide;
(b) contacting said cell with a dsRNA in the presence of a candidate compound; and
(c) monitoring a dsRNA-related phenotype in said cell in the presence of said candidate compound relative to said phenotype in the absence of said candidate compound, wherein an alteration in said phenotype identifies said candidate compound as a candidate compound that enhances RNAi.

32. An isolated ERI-1 polypeptide comprising an amino acid sequence having at least 90% identity to the amino acid sequence of SEQ ID NO:2, wherein said polypeptide inhibits RNAi.

33. An isolated nucleic acid molecule comprising a nucleotide sequence having at least 90% identity to the nucleotide sequence encoding SEQ ID NO:2, wherein expression of said nucleic acid molecule in an organism inhibits RNAi in said organism.

34. A vector comprising the isolated nucleic acid molecule of claim 26.

35. A host cell comprising the vector of claim 34.

36. An antibody that specifically binds to an ERI-1 polypeptide.

37. An organism comprising a mutation in an eri-1 nucleic acid sequence, wherein said mutation enhances RNAi in said organism.

38. The organism of claim 37, wherein said organism is a nematode.

39. The organism of claim 37, wherein said organism is a mammal.

40. The organism of claim 30, wherein said organism is a plant.

41. An isolated nucleobase oligomer comprising a duplex comprising at least eight but no more than thirty consecutive nucleobases of an eri-1 nucleic acid, wherein said duplex when contacted with an eri-1 expressing cell, reduces eri-1 transcription or translation.

42. The oligomer of claim 41, wherein said duplex comprises a first domain comprising between 21 and 29 nucleobases and a second domain that hybridizes to said first domain under physiological conditions, wherein said first and second domains are connected by a single stranded loop.

43. The oligomer of claim 41, wherein said loop comprises between 6 and 12 nucleobases.

44. The oligomer of claim 41, wherein said loop comprises 8 nucleobases.

45. The oligomer of claim 41, wherein said oligomer reduces the level of expressed ERI-1 polypeptide.

46. A nucleobase oligomer comprising a first region, wherein said first region comprises at least eight but no more than thirty consecutive nucleobases corresponding to an eri-1 nucleic acid molecule, and a second region, wherein said second region comprises at least eight but no more than thirty consecutive nucleobases complementary to said first region, and said oligomer when contacted with an eri-1-expressing cell, reduces eri-1 transcription or translation.

47. The nucleobase oligomer of claim 46, wherein said nucleobase oligomer is an shRNA.

48. The nucleobase oligomer of claim 46, wherein said nucleobase oligomer comprises at least one nucleic acid modification.

49. The nucleobase oligomer of claim 46, wherein said modification is a modified sugar, nucleobase, or internucleoside linkage.

50. The nucleobase oligomer of claim 46, wherein said modification is a modified internucleoside linkage selected from the group consisting of phosphorothioate, methylphosphonate, phosphotriester, phosphorodithioate, and phosphoselenate linkages.

51. The nucleobase oligomer of claim 46, wherein said nucleobase oligomer comprises at least one modified sugar moiety.

52. The nucleobase oligomer of claim 46, wherein said nucleobase oligomer comprises RNA residues.

53. The nucleobase oligomer of claim 52, wherein said RNA residues are linked together by phosphorothioate linkages.

54. An expression vector encoding a nucleobase oligomer comprising a duplex comprising at least eight but no more than thirty consecutive nucleobases of an eri-1 nucleic acid, wherein said duplex, when contacted with an eri-1-expressing cell, reduces eri-1 transcription or translation.

55. An expression vector encoding a nucleobase oligomer comprising a first region, wherein said first region comprises at least eight but no more than thirty consecutive nucleobases corresponding to an eri-1 nucleic acid molecule, and a second region, wherein said second region comprises at least eight but no more than thirty consecutive nucleobases complementary to said first region, and said oligomer when contacted with an eri-1-expressing cell, reduces eri-1 transcription or translation.

56. The expression vector of claim 54 or 55, wherein a nucleic acid sequence encoding said nucleobase oligomer is operably linked to a promoter.

57. The expression vector of claim 56, wherein said promoter is the U6 PolIII promoter, polymerase III H1 promoter.

58. A cell comprising the expression vector of claim 54 or 55.

59. The cell of claim 58, wherein said cell is a transformed human cell that stably expresses said expression vector.

60. The cell of claim 58, wherein said cell is in vivo.

61. The cell of claim 58, wherein said cell is a human cell.

62. The cell of claim 58, wherein said cell is a neoplastic cell.

63. A transgenic organism expressing a nucleic acid sequence encoding an eri-1 nucleobase oligomer, wherein said nucleobase oligomer inhibits the expression of an endogenous eri-1 nucleic acid sequence.

64. The organism of claim 63, wherein said organism is a mammal.

65. The organism of claim 63, wherein said organism is a nematode.

66. The organism of claim 63, wherein said organism is a plant.

67. A method for enhancing RNAi in an organism, said method comprising contacting said organism with a nucleobase oligomer of claim 46 in an amount sufficient to enhance RNAi.

68. The method of claim 67, wherein said organism is a plant.

69. The method of claim 67, wherein said organism is a mammal.

70. The method of claim 67, wherein said organism is a pathogen, selected from the group consisting of a bacteria, a virus, a fungus, an insect, or a nematode.

71. The method of claim 67, wherein said nucleobase oligomer is an siRNA or an shRNA.

72. A pharmaceutical composition comprising an eri-1 nucleobase oligomer and an excipient.

73. A double-stranded RNA corresponding to at least a portion of an eri-1 nucleic acid molecule of an organism, wherein said double-stranded RNA is capable of decreasing the level of ERI-1 polypeptide encoded by an eri-1 nucleic acid molecule.

74. An antisense nucleic acid molecule, wherein said antisense nucleic acid molecule is complementary to at least six nucleotides of an eri-1 nucleic acid molecule, and wherein said antisense nucleic acid molecule is capable of decreasing expression of an ERI-1 polypeptide from an eri-1 nucleic acid molecule.

75. A method for identifying an siRNA having enhanced RNAi activity, said method comprising:
(a) contacting a test siRNA with an ERI-1 polypeptide under conditions suitable for RNA degradation;
(b) measuring the amount of undegraded test siRNA relative to a control siRNA known to be degraded under similar conditions, wherein increased resistance to degradation indicates that said test siRNA has enhanced RNAi activity.

76. An siRNA capable of inducing enhanced RNAi, said siRNA comprising a 3′ terminus having at least 2 cytosine or guonosine bases, such that said siRNA resists degradation by ERI-1.

77. An isolated eri-1 inhibitory nucleic acid comprising at least a portion of a naturally occurring eri-1 nucleic acid molecule of an organism, or its complement, where the eri-1 nucleic acid encodes a polypeptide selected from the group consisting of any or all of the following T07A9.5, BC035279, T04799, BC035279, BAB02568.1, NP_{—}566502.1, T04799, NP_{—}921413.1, NP_{—}179108.1, AAL31944.1, AAL84996.1, CAB36522.1, CAB79531.1, AAK98687.1, AAP53700.1, NP_{—}499887.1, NP_{—}500418.1, NP_{—}741292.1, NP_{—}741293.1, T28707, NP_{—}508415.1, NP_{—}497750.1, NP_{—}507742.1, T15066, AAB94148.1, T29900, AAB09126.1, AAK39277.2, NP_{—}741293.1, T32575, AAK39278.1, T28707, NP_{—}508415.1, Q10905, YWO2_CAEEL, T30086, AAA82440.1, AAP57300.1, NP_{—}741293.1, NP_{—}507945.1, T19258, NP_{—}505050.1, T32575, AAK39278.1, T26693, CAA20983.1, T33294, AAC17749.1, AK064632.1, AP002897.2, AK103348.1, AK062026.1, AY105868.1, NM_{—}112377.1, AF419612.1, AF419612, AY079112.1, AP002862.2, AP000815.1, AP003103.2, AK120298.1, NM_{—}191971, AY112398.1, AC146855.5, AY105981.1, NM_{—}117213.2, AF291711.1, AF291711, AK120333.1, AK106560.1, AB019236.1, AK122166.1, NM_{—}184142.1, NM_{—}196431.1, and AC093544.8, or an ortholog of any or all of these eri-1 nucleic acid molecules, where the eri-1 inhibitory nucleic acid comprises at least a portion of a naturally occurring eri-1 nucleic acid molecule, or is capable of hybridizing to a naturally occurring eri-1 nucleic acid molecule, and decreases expression from a naturally occurring eri-1 nucleic acid molecule in the organism.


SEQUENCE LISTING (35 sequences)

SEQ ID NO: 1 - DNA, 4175 bp, C. elegans
atgagcgcagatgagccgtctccagaagacgaaaaatatctggaaagtctacgagatttgctgaaaatcagccaagaattcgatgcaagtaacgcaaaacagaatgacgaaccagaaaaaacagcagttgaagtggaatcggctgaaaccagaaccgacgaatctgaaaaatcaatcgatattccacgtgaacagcaacttttaccctccgaacgtgttgagcctttaaaatcaatggtggaaccagaatacgtgaaaaaagtgataaggcaaatggatacaatgactgctgaacagctaaaacaagcgttgatgaagattaaagtgtccacagggtaaaattatgctaaattcattcaaacttcttattttcctaacagaacccaatttcgtacatttaagatatagctttgcttcatttttgatagttttgttaaaaagaaacagcattttttgaatcattttgcgttaaatatagtcatccgtaaaagaaataatgtaattttcttactaaaatttcgaaattaacttaaattataactctaatttcagcggaaacaagaaaactcttcgaaaacgggttgcacaatattatcgaaaggaaaatgcattattgaatcgaaaaatggagccgaatgccgataaaactgcacgcttttttgactatttaattgtatgattttcagaatttctggaaaagaaatactttagttattaattatttcaggcaattgatttcgagtgtacgtgcgtcgagattatatacgattacccacacgaaatcatcgaattaccggcagttttgatcgacgttcgggaaatgaaaattgtaggattttttcatctttgaaaatggaatatgtcgaattacgcatgtcgtgtactccccgcggacaaggattgaacagttctcagttgaaattcttgattttggatttttcctctggggtttcggctcgaattttatttttttttttcgtgttttcccgcaagaaaatcattttttccgggaaaaggtgacaaacgtttcgtcatcctatcgaaatctggaatataaaaatgtattcaacaaaaaacgtttcaaatacaacatttttcgtcttttttgggtttcttatttagctataaaatttgtttcgcttgtgaagaaaatattggaaaaaaatagaaaaatccttctcccatttttcaatttaaggcagatggaagttctttggtgtattatcaagttataaaaactgactaaactgaaagctttttttgttttgttttgtttatttattttaggaatataaataaaaaacgaaaaatatataactttataatatccatcgtcaaaataagcatatttaatttccagatcagcgagtttcgaacctatgtccgacctgtcagaaatccgaagctcagcgaattttgtatgcagtttaccagtaagacactaatgtcatttttaaaatgtaaaccctgttgtagaaattgcccaagaaacagtcgatgctgctccatatttccgagaggctcttcaacggctgtacacatggatgcgaaaattcaatctcggacagaaaaattccagattcgcatttgttactgatgggtaaggaatcgaagacgattttttgttcactaaaacttcaaagttgtgcataacgaactattcagacctcacgatatgtggaaattcatgcaatttcaatgccttttatcgaatattcgaatgccccatatgttccgaagttttatcaacatcaaaaagacattcaaagagaaattcaatggacttatcaagggaaatggagtaagtttttaaggttcaaattttaattaaaacctcaattctcttcagaaatccggaattgaaaacatgttagaacgtctggatctgtcgtttgttggaaataaacatagtggattggatgatgcaacgaatattgcagcgattgcgattcaaatgatgaagttgaaaattgagttgagaattaatcagaaatgctcgtataaggagaatcaacggagtgctgcgcgaaaggatgaagagcgagaggttcgaaacagactacgtcgttttcacccgatgggttgactctcccattcggtctcgggtgtcaacaagtgattccatgttttgaaaaaagctcttccctcaacccttaaaatttcgatatgaaattgtttcagtgcatttttcactaatttggagacctgttgaattgctctaaatgtttttttgcaaagttcaatgtttttcagaacggttataagtaggtgaacactctgggagatttttggacttttttttgttgtgaaaatctgaacatttttggatcaagtggagtgaagaaactttcagatttttttcaaaaaattacaaatttttgaaaaatctcccagagtgttcacctacttctaaacgttctgaaaaaacattaaagtttgaaaaaaacgcatttggagcaattctacaggcctccaaagtagtgaaaacaggcactgaaacaattccagatcgaaatgaagtctgacgataaaatggaaacctttccttcttgttaggaaaattaggaaatagttcatcacaaatttcaattaaaaagttattaaatttttcagttagaagacgctgcaaacgtggatcttacatcagtcgatatctctcgtcgtgattttcaactttggatgcgtcgtttaccactgaaactgtcctctgtgacacgacgagagttcataaatgaagagtatttggattgtgattcatgtgatgatttgactgatgataaggtgaaacatctacattcttgtgatatttatgaaattttcgatgaaaaaacttcagctagttttacagattcaaagtgtttgatttgctaggcgaattttggcaaaagtatacttattgatgtaaacattttccctatcaattttgtgtcttagccaacgaatttccaaattttatttcaaaagtaaaattccatgtcaaattttacatttttagtttaaaaattatgtttcttccaagcccacatttttcattgaaactggtatagttgtatttgattcgccccgaataaataattttctgtttccatgttttatacacacaaaaactcaaaattttcagaacgacgaggcggcattccaggaaaaaatggcaattcgagagtatttggagaataaacagacggaagatttcgcgaaaattgctgctgagcggggaatttttaaaattgggtaatggactttggatggaattttgaacaaaaaaatctagtttctggcgttttcagtgaaataaaatcatatcaaactgcaagaccgataatagaggatgatgacgtggatgttgaaagtgaagaggaagattatggaactgaatttgaaatgttggaagttgtggtgagaatgttgagaaaaaacacttcaaaaatatcaattttgtttttaaaaatcaacgccacgattcgatgaatcgaaattcaaaaattctccgaaaaagcggaaaactctgcttttgaaatcagaaatttgcaatgttttccgggataatcgaaaatttctttcaaaaaaaaattaatgtaggtataaatttcagattcatcgtggtgtggattttaaaaatccgcgagagaaaaaaattctgaataactaagcttttcgattttcgtataatacaaaaccgaacttttattttgttttcaaattttaataaaattcgagcaaaaaaccaaatcgatttggagttttaataattaattttttcgttcaggaaagaatgcctccagttagttctacgttacacactgaagtcgatttagatgctgtatgggaacgagatggtggaagtgattctgaaagaggtacgtcgagggaattgcaatatggaaattgcattagaaaaagcttgaaattctgtttgattaaaatgtcaattcaattgctcccaaagtttaaattattgaatagcacttaaaaaacataattgtgaaaattcagaaaacctctcaaatgctccaagtctccacgagtttccatcgtcatccacatcatcaccacatgccacgtcagaacatgtgacgtcatcatcaccacttcatatcgacgatgacgtggaccgcgtgttgaatgcgccgcccaaaaattcgttggcatcgtcttcaaatcgatctagtttctag

SEQ ID NO: 2 - protein, 582 aa, C. elegans
MSADEPSPEDEKYLESLRDLLKISQEFDASNAKQNDEPEKTAVEVESAETRTDESEKSIDIPREQQLLPSERVEPLKSMVEPEYVKKVIRQMDTMTAEQLKQALMKIKVSTGGNKKTLRKRVAQYYRKENALLNRKMEPNADKTARFFDYLIAIDFECTCVEIIYDYPHEIIELPAVLIDVREMKIISEFRTYVRPVRNPKLSEFCMQFTKIAQETVDAAPYFREALQRLYTWMRKFNLGQKNSRFAFVTDGPHDMWKFMQFQCLLSNIRMPHMFRSFINIKKTFKEKFNGLIKGNGKSGIENMLERLDLSFVGNKHSGLDDATNIAAIAIQMMKLKIELRINQKCSYKENQRSAARKDEERELEDAANVDLTSVDISRRDFQLWMRRLPLKLSSVTRREFINEEYLDCDSCDDLTDDKNDEAAFQEKMAIREYLENKQTEDFAKIAAERGIFKIGEIKSYQTARPIIEDDDVDVESEEEDYGTEFEMLEVVERMPPVSSTLHTEVDLDAVWERDGGSDSERENLSNAPSLHEFPSSSTSSPHATSEHVTSSSPLHIDDDVDRVLNAPPKNSLASSSNRSSF

SEQ ID NO: 3 - protein, 448 aa, C. elegans
MSADEPSPEDEKYLESLRDLLKISQEFDASNAKQNDEPEKTAVEVESAETRTDESEKSIDIPREQQLLPSERVEPLKSMVEPEYVKKVIRQMDTMTAEQLKQALMKIKVSTGGNKKTLRKRVAQYYRKENALLNRKMEPNADKTARFFDYLIAIDFECTCVEIIYDYPHEIIELPAVLIDVREMKIISEFRTYVRPVRNPKLSEFCMQFTKIAQETVDAAPYFREALQRLYTWMRKFNLGQKNSRFAFVTDGPHDMWKFMQFQCLLSNIRMPHMFRSFINIKKTFKEKFNGLIKGNGKSGIENMLERLDLSFVGNKHSGLDDATNIAAIAIQMMKLKIELRINQKCSYKENQRSAARKDEERELEDAANVDLTSVDISRRDFQLWMRRLPLKLSSVTRREFINEEYLDCDSCDDLTDDKVKHLHSCDIYEIFDEKTSASFTDSKCLIC

SEQ ID NO: 4 - DNA, 2195 bp, Homo sapiens
ccgccgccgcgggaacgcgagcccggtaatttttcaacggagaaaggcgaggctttcgggctctgcagagtgagagttagcaagtgtccggctccagcaactctcctctggcgtgacagccggcatggaggatccacagagtaaagagcctgccggcgaggccgtggctctcgcgctgctggagtcgccgcggccggagggcggggaggagccgccgcgtcccagtcccgaggaaactcaacagtgtaaatttgatggccaggagacaaaaggatccaagttcattacctccagtgcgagtgacttcagtgacccggtttacaaagagattgccattacgaatggctgtattaatagaatgagtaaggaagaactcagagctaagctttcagaattcaagcttgaaactagaggagtaaaggatgttctaaagaagagactgaaaaactattataagaagcagaagctgatgctgaaagagagcaattttgctgacagttattatgactacatttgtattattgactttgaagccacttgtgaagaaggaaacccacctgagtttgtacatgaaataattgaatttccggttgttttactgaatacgcatactttagaaatagaagacacgtttcagcagtatgtaagaccagagattaacacacagctgtctgatttctgcatcagtctaactggaattactcaggatcaggtagacagagctgataccttccctcaggtactaaaaaaagtaattgactggatgaaattgaaggaattaggaacaaagtataaatactcacttttaacagatggttcttgggatatgagtaagttcttgaacattcagtgtcaactcagcaggctcaaataccctccttttgcgaaaaagtggatcaatattcggaagtcatatggaaatttttacaaggttcctagaagccaaaccaaactgacaataatgcttgaaaaattaggaatggattatgatgggcggcctcactgtggtcttgatgactctaagaatatcgcccgaatagcagttcgaatgcttcaggatgggtgtgaactccgaatcaacgagaaaatgcatgcaggacagctaatgagtgtgtcctcttccttaccaatagagggcactccaccaccacaaatgccacattttagaaagtaacaacagttttgtgtgtggatcattccaattgaagttgctatgaagaggtagcagatgaatctcattgaattagtcctgtagtgcaaactttaagcaccttaaaacatttaaaatcttattacaggtgatagagatagatacatgtatgtgaacagattttgtaggaaggcatactgaattctttgtcaccagcacttttgatatgaacagtattcgttacatagtaacagttcctgcttacaactgaattttataatttaaggtgttcaagatatattctttttggttttaaaatgcaaaatcttattggctgttctgttgaatgtcatatcttactggtgtttaaatatgtaatgtgtttctttattaacatcactagatgaaaccatatcttaaaatgcagaaatgattggaaggtagatcttatctagcctttggatttcaagaatatcatagtccttttgattttcaaagtttatatgtgaagttcaccatgtatgtggtgaatttcgtaaggtacttggtatacatatctgcctatgtttcttttcaactcataattggaagaattatgatggattatagggtttggttaaaaatccagttactgaaggaattaatgaaaacgtagaagaaagtactaaaaggaatatcataagggctgtagctcaaacttcataatacataaatcactggggtctttttggatttggttgtttgattcgccttccttttttgacatatgtatgccttaattcttaaatctgagggaccatgctttgaaatagactgaaaattaagggtcaccacctaattttactttgtattcagtatcgtaagtgaggttaataaagtcaatactttctaccatatattacgtttttgttattaaaaaacttcattggccactagtgaagttagtcaataaaagacttgtttttctgaaaaaaaaaaaaaaaaaaaaaaaaaaaaaaaaaaaaaaaaaaaaaaaaaaaaaaaaaaaaaaaaaaaaaaaaaaaaa

SEQ ID NO: 5 - protein, 349 aa, Homo sapiens
MEDPQSKEPAGEAVALALLESPRPEGGEEPPRPSPEETQQCKFDGQETKGSKFITSSASDFSDPVYKEIAITNGCINRMSKEELRAKLSEFKLETRGVKDVLKKRLKNYYKKQKLMLKESNFADSYYDYICIIDFEATCEEGNPPEFVHEIIEFPVVLLNTHTLEIEDTFQQYVRPEINTQLSDFCISLTGITQDQVDRADTFPQVLKKVIDWMKLKELGTKYKYSLLTDGSWDMSKFLNIQCQLSRLKYPPFAKKWINIRKSYGNFYKVPRSQTKLTIMLEKLGMDYDGRPHCGLDDSKNIARIAVRMLQDGCELRINEKMHAGQLMSVSSSLPIEGTPPPQMPHFRK

SEQ ID NO: 6 - DNA, 32 bp, C. elegans
ttcgataaagtgcctgtttttttttcgataaa

SEQ ID NO: 7 - protein, 190 aa, C. elegans
YLIAIDFECTCVEIIYDYPHEIIELPAVLIDVREMKIISEFRTYVRPVRNPKLSEFCMQFTKIAQETVDAAPYFREALQRLYTWMRKFNLGQKNSRFAFVTDGPHDMWKFMQFQCLLSNIRMPHMFRSFINIKKTFKEKFNGLIKGNGKSGIENMLERLDLSFVGNKHSGLDDATNIAAIAIQMMKLKIE

SEQ ID NO: 8 - protein, 187 aa, Homo sapiens
YICIIDFEATCEEGNPPEFVHEIIEFPVVLLNTHTLEIEDTFQQYVRPEINTQLSDFCISLTGITQDQVDRADTFPQVLKKVIDWMKLKELGTKYKYSLLTDGSWDMSKFLNIQCQLSRLKYPPFAKKWINIRKSYGNFYKVPRSQTKLTIMLEKLGMDYDGRPHCGLDDSKNIARIAVRMLQDGCE

SEQ ID NO: 9 - protein, 183 aa, Zea mays
YFVVIDFEATCDKVNNPFPQEIIEFPSVLVNSATGKLEECFQTYVRPTYHQFLTDFCKELTGIQQIQVDRGVPLGEALLMHDKWLEDKGIKNTNFAIVTWSNWDCRIMLESECRFKRIRKPPYFNRWINLRVPFQEVYGDVRCNLKEAVQLAGLTWEGRAHCGLDDARNTARLLALLMHRGFK

SEQ ID NO: 10 - protein, 187 aa, Oryza sativa
HFVVVDFEATCERGRRIYPQEIIEFPAVLVDAATGRLVSAFRAYVRPRHHPRLTDFCRELTGIAQGDVDAGVGLAEALLRHDEWLRAAGVVEGGGRFAVVTWGDADCRTMLEQECRFKGIAKPAYFDRWVDLRVHFEAAFGGGGQRVKLQEAVRAAGLEWVGRLHCGLDDACNTARLLVELLRRGVP

SEQ ID NO: 11 - protein, 184 aa, Arabidopsis thaliana
FLVIDLEGKVEILEFPILIVDAKTMEVVDLFHRFVRPTKMSEQAINKYIEGKYGELGVDRVWHDTAIPFKQVVEEFEVWLAEHDLWDKDTDWGLNDAAFVTCGNWDIKTKIPEQCVVSNINLPPYFMEWINLKDVYLNFYGREARGMVSMMRQCGIKLMGSHHLGIDDTKNITRVVQRMLSEGA

SEQ ID NO: 12 - protein, 13 aa, C. elegans
YLIAIDFECTCVE

SEQ ID NO: 13 - protein, 13 aa, C. elegans
FVTDGPHDMWKFM

SEQ ID NO: 14 - protein, 12 aa, C. elegans
GNKHSGLDDATN

SEQ ID NO: 15 - protein, 13 aa, C. briggsae
NFQAIDFECTCVE

SEQ ID NO: 16 - protein, 13 aa, C. briggsae
FVTDGPHDMWKFM

SEQ ID NO: 17 - protein, 12 aa, C. briggsae
GNKHSGLDDARN

SEQ ID NO: 18 - protein, 13 aa, Homo sapiens
YICIIDFEATCEE

SEQ ID NO: 19 - protein, 13 aa, Homo sapiens
LLTDGSWDMSKFL

SEQ ID NO: 20 - protein, 12 aa, Homo sapiens
GRPHCGLDDSKN

SEQ ID NO: 21 - protein, 13 aa, Mus musculus
YICIIDFEATCEE

SEQ ID NO: 22 - protein, 13 aa, Mus musculus
ILTDGSWDMSKFL

SEQ ID NO: 23 - protein, 12 aa, Mus musculus
GRPHSGLDDSKN

SEQ ID NO: 24 - protein, 13 aa, X. laevis
YICVIDFEATCEA

SEQ ID NO: 25 - protein, 13 aa, X. laevis
ILTDGSWDMSKFL

SEQ ID NO: 26 - protein, 12 aa, X. laevis
GRLHSGLDDSKN

SEQ ID NO: 27 - protein, 13 aa, Danio rerio
YICVVDFEATCEE

SEQ ID NO: 28 - protein, 13 aa, Danio rerio
FLTDGSWDMGKFL

SEQ ID NO: 29 - protein, 12 aa, Danio rerio
GRPHCGLDDSRN

SEQ ID NO: 30 - protein, 13 aa, S. pombe
YLLIVDVEATCEE

SEQ ID NO: 31 - protein, 13 aa, S. pombe
WACDGPWDMASFL

SEQ ID NO: 32 - protein, 12 aa, S. pombe
GSEHRGIDDARN

SEQ ID NO: 33 - protein, 13 aa, Homo sapiens
KCVAIDCEMVGTG

SEQ ID NO: 34 - protein, 13 aa, Homo sapiens
VVGHALHNDFQAL

SEQ ID NO: 35 - protein, 11 aa, Homo sapiens
QHGHSSVEDAT